(19) United States
(12) Patent Application Publication
(10) Pub. No.: US 2012/0148516 A1
(43) Pub. Date: Jun. 14, 2012

(54) Tandem Hypochlorite-Barrier Repair Therapy for the Treatment of Eczema

(75) Inventors: Douglas Abel, San Diego, CA (US); Mark W. Trumbore, Westford, MA (US); Ronald M. Gurge, Franklin, MA (US)

(73) Assignee: Precision Dermatology, Inc., Cumberland, RI (US)

(21) Appl. No.: 13/313,468

(22) Filed: Dec. 7, 2011

Related U.S. Application Data
(60) Provisional application No. 61/421,696, filed on Dec. 10, 2010.

(51) Int. Cl. A61K 8/20; A61P 17/06; A61P 31/04; A61P 31/12; A61P 31/10; A61P 17/00; A61P 31/22

(52) U.S. Cl. 424/62

(57) ABSTRACT

Described herein are compositions, kits, and methods for the treatment of atopic dermatitis and related dermatological disorders. The invention encompasses a two-step method of treating a condition, wherein the first step involves applying an inventive bleach composition to the skin, and the second step involves applying an inventive barrier repair composition to the skin.

### RELATED APPLICATIONS

This application claims the benefit of priority to U.S. Provisional Patent Application Ser. No. 61/421,696, filed Dec. 10, 2010, the contents of which are hereby incorporated by reference.

### BACKGROUND

Atopic dermatitis (AD), commonly referred to as eczema, is a disease characterized by dry, cracked, itchy, and inflamed skin, often presenting on greater than 10% of the body surface area. It accounts for 10-20% of all visits to dermatologists and affects approximately 3% of the US population, most of whom are children. The condition is characterized by intense pruritus (itch) and a course marked by exacerbations and remissions. Higher transepidermal water loss (TEWL) has also been noted in dry skin atopic patients; TEWL is indicative of a disturbed barrier function, and it has been correlated to pruritus intensity in patients. Further, the compromised skin barrier allows excessive water loss through the epidermal layer of the skin and the potential penetration of allergens.

Environmental factors, such as psychological stress, climate (e.g., low humidity caused by cold winters and central heating), and exposure to irritants and allergens determine the course of the disease. The defective barrier function associated with AD can also make atopic patients more prone to irritant contact dermatitis, since ordinary soaps and detergents often irritate the skin. Exposure to hard water, especially to calcium salts in domestic water, has been found to be associated with a higher prevalence of atopic eczema in primary-school children.

Another potential triggering factor for AD is the colonization of the skin with microorganisms, such as Staphylococcus aureus and Malassezia. S. aureus can release superantigenic exotoxins, which produce a massive release of cytokines Staphylococcus enterotoxin B also induces eczema when applied to uninvolved atopic and normal skin, while the severity of AD has been reported to correlate linearly with S. aureus counts.

A doctor has three main goals in designing a treatment regime for the patient: healing the skin and keeping it healthy, preventing flare ups, and treating symptoms when they do occur. Proper skin care and moisturizing ointments are the mainstays of topical treatment. Moisturizers which improve barrier function have been reported which reduce the prevalence of AD and can reduce the associated symptoms.

In addition to moisturizers, a variety of medications may be prescribed to help manage the condition. Topical steroids are the first-line treatment for atopic dermatitis flares because they are effective at reducing the inflammation caused by this disease. Immunomodulators (calcineurin inhibitors, such as tacrolimus and pimecrolimus) may also be prescribed. Immunomodulators change some of the functions of the immune system that cause atopic dermatitis without suppressing the whole immune system. Other immune-suppressing medications being investigated as a treatment for atopic dermatitis include: cyclosporine, interferon, methotrexate, and azothiaprine. Another mechanism of treatment includes the use of oral antihistamines, such as diphenhydramine or hydroxyzine. Oral antihistamines are used to treat itch associated with atopic dermatitis; however, they can cause sleepiness and may not help in all cases of atopic dermatitis.

For mild cases of atopic dermatitis, an over-the-counter formulation of coal tar is often used. Coal tar has long been a treatment for a variety of skin conditions. Shampoos and soaps containing coal tar can help with mild cases of atopic dermatitis. Coal tar tends to work better on thickened skin that is not scaly, and the early symptoms of itching. However, coal tar can be irritating to already inflamed skin. Coal tar is used for mild cases of atopic dermatitis only.

For more severe flares of atopic dermatitis—for example if the rash covers a large part of the body or face—oral corticosteroids, such as prednisone, prednisolone, and medrol, may be used. Long-term use of oral steroids has numerous side effects, including weight gain, thinning of the bones, and suppression of the immune system; consequently, though they may clear atopic dermatitis well, the side effects are too risky to warrant using them as a first-line treatment. To avoid these side effects, but still benefit from the medication, oral steroids are often prescribed for a short course (e.g., five days) to calm the rash. Topical steroids can then be used on the remaining rash.

As discussed above, atopic dermatitis reduces the skin's natural defenses, making it easier for skin to become infected. If the skin becomes infected, antibiotics are often prescribed. Antibiotics, such as cefadroxil or cephalexin, are often prescribed at the first sign of infection.

Treatment with aqueous hypochlorite compositions has been shown to decrease the microbial burden associated with atopic dermatitis, resulting in an improvement in symptoms. Skin and wound cleansers containing bleach have also been shown to reduce microbial skin contamination. Incorporation of bleach into leave-on products intended for long term skin contact allows for the potential to provide additional long-term treatment benefits, such as improved moisturization and control of transepidermal water loss, not possible with products intended for transient skin contact.

Hypochlorite salts are inherently unstable in aqueous solution. The decomposition rate of hypochlorite in water is dependent on concentration, temperature, and pH. High temperatures and acidic pHs greatly accelerate the rate of decomposition, as does the presence of metal ions. The normal shelf-life of bleach solutions is approximately six months at a pH between 12 and 13.5. Additionally, due to the high pH and oxidative potential of bleach, bleach solutions are frequently irritating to the skin. In addition, due to its oxidative potential, topical bleach treatment can be associated with changes in the color of clothing and hair.

U.S. Pat. No. 7,622,434 describes gel compositions of sodium hypochlorite suitable for topical application, but makes no mention of their possible utility in the treatment of atopic dermatitis or the possibility of their use in combination with barrier repair treatments. US published patent applications 2005/0196462 and 2007/0196357 describe topical formulations of oxidative reductive potential (ORP) water solutions containing hypochlorite and their use in the topical treatment of disease. While US 2007/0196357 does describe the ability of the ORP formulations to kill Staphylococcus aureus, no mention is made in either US 2005/0196462 or US 2007/0196357 of the utility of the solutions in the treatment of atopic dermatitis, or their use in conjunction with barrier repair compositions. U.S. Pat. No. 6,589,568 describes the sequential topical application of dilute bleach formulations followed by topical application of a second “body lotion” formulation. However, U.S. Pat. No. 6,589,568 specifically describes the combination as useful for subcutaneous treatment and makes no mention of atopic dermatitis, which is a disease of cutaneous tissue. Likewise, U.S. Pat. No. 6,589,568 does not recognize the importance of rapid inactivation of hypochlorite once applied topically, thereby reducing the irritation and clothing color change associated with topical bleach application; this patent also does not address repairing damaged barrier function.

There exists a need for methods of treatment that address the irritation suffered by patients after application of topical bleach solutions.

### SUMMARY OF THE INVENTION

In one embodiment, the invention relates to a kit, wherein the kit comprises a bleach composition; and a barrier repair composition.

In one embodiment, the present invention relates to a method of treating a condition of a subject in need thereof, comprising the steps of:

applying to an affected area of the subject an effective amount of a bleach composition; and

applying to the affected area of the subject an effective amount of a barrier repair composition.

In one embodiment, the present invention relates to a method of treating a condition of a subject in need thereof, comprising the steps of:

applying to an affected area of the subject an effective amount of a bleach composition; and

applying to the affected area of the subject an effective amount of a barrier repair composition,

thereby treating and moisturizing the affected area.

In one embodiment, the present invention relates to a method of treating a condition of a subject in need thereof, comprising the steps of:

applying to an affected area of the subject an effective amount of a bleach composition; and

applying to the affected area of the subject an effective amount of a barrier repair composition,

thereby treating and hydrating the affected area.

In one embodiment, the present invention relates to any one of the above-mentioned methods, wherein the condition is atopic dermatitis, contact dermatitis, xerotic eczema, seborrhoeic dermatitis, psoriasis, dyshidrosis, discoid eczema, venous eczema, dermatitis herpetiformis, neurodermatitis, autoeczematization, herpes simplex I, other topical herpes or viral infections, common cold sores and fever blisters, ringworm, impetigo, or other viral, fungal, or bacterial infections of the skin.

In one embodiment, the present invention relates to any one of the above-mentioned methods, wherein the condition is eczema.

In one embodiment, the present invention relates to any one of the above-mentioned methods, wherein the condition is atopic dermatitis.

In one embodiment, the present invention relates to any one of the above-mentioned methods, wherein the barrier repair composition is applied to the affected area from about 1 minute to about 30 minutes after the bleach composition is applied.

### BRIEF DESCRIPTION OF THE FIGURES

FIG. 1 tabulates the components present in various barrier repair emulsions of the invention.

FIG. 2 depicts a graph showing the decrease in transepidermal water loss following treatment with various barrier repair emulsions.

FIG. 3 tabulates the components present in various bleach compositions of the invention.

FIG. 4 tabulates the reduction in available hypochlorite following exposure to a barrier repair composition (N.P.=Not Performed).

### DETAILED DESCRIPTION OF THE INVENTION

### Overview

In certain embodiments, the invention relates to a method of treating AD comprising the sequential use of a topical hypochlorite composition followed by the use of a topical barrier repair composition. In certain embodiments, this method of treatment controls the symptoms of AD while reducing the irritation and bleaching potential of topical hypochlorite therapy. In certain embodiments, application of topical hypochlorite compositions reduces the number of skin associated bacteria, yeast, and fungi. In certain embodiments, application of barrier repair compositions hydrates skin and restores barrier function. In certain embodiments, both kinds of compositions are individually useful in the control of the symptoms of AD. In certain embodiments, the invention relates to a method of treating AD in a subject in need thereof, comprising the steps of first applying a topical hypochlorite composition, and then applying a topical barrier repair composition several minutes later. In certain embodiments, this method of treatment provides greater therapeutic efficacy in the treatment of atopic dermatitis than is achieved with either therapy alone.

In certain embodiments, the invention relates to topical bleach compositions, wherein the bleach composition may be in the form of a solution, gel, lotion, cream, foam, ointment, paste or spray. In certain embodiments, the bleach composition may contain from about 0.001 to about 0.5% hypochlorite. In certain embodiments, the hypochlorite may be the salt of any Group I or II alkali or alkaline earth element. In certain embodiments, the bleach composition is stable. In certain embodiments, when applied to the skin a bleach composition of the invention reduces the number of skin-associated bacteria, yeasts, and fungi, improves skin moisture levels, and is non-irritating and non-drying. In certain embodiments, the invention relates to a bleach composition that is suitable for the treatment of atopic dermatitis.

In certain embodiments, the invention relates to topical barrier repair composition, wherein the barrier repair composition may be in the form of a solution, gel, lotion, cream, foam, ointment, paste, or spray. In certain embodiments, the barrier repair composition may reduce the transepidermal water loss of the area to which it is applied.

In certain embodiments, the invention relates to a method of treating a condition in a subject in need thereof, comprising the step of applying a bleach composition to an affected area at least once per day. In certain embodiments, the invention relates to any one of the aforementioned methods, further comprising the step of applying a barrier repair composition to the affected area after a period of time ranging from about 1 to about 30 minutes. In certain embodiments, the barrier repair composition is applied without washing off the bleach composition. In certain embodiments, applying the barrier repair composition rapidly inactivates the hypochlorite, thereby preventing discoloration of clothing and reducing skin irritation. In certain embodiments, the methods of treatment described herein improve the symptoms of atopic dermatitis by reducing the number of skin associated bacteria, yeasts and fungi, reducing transepidermal water loss, and improving skin moisture levels and barrier function.

In one embodiment, the compositions do not contain a volatile lower alcohol. In certain embodiments, the invention relates to a composition that does not comprise a steroid.

### Moisturization and Irritation

Topical compositions of hypochlorite salts are known to be irritating and lack the ability to hydrate skin, generally. These two negative attributes can lead to reduced patient compliance with its concomitant impact on therapeutic benefit. In one embodiment, the inventive compositions of hypochlorite salts are no more irritating than vehicle control. In one embodiment, the inventive compositions of hypochlorite salts demonstrate similar levels of erythema as intact untreated skin. In one embodiment, the inventive compositions have the ability to moisturize skin.

In certain embodiments, the methods of treatment described herein also address the problems associated with irritation and lack of moisturization in existing treatments of AD.

### Exemplary Compositions

Exemplary Bleach Compositions

In one embodiment, the invention relates to a bleach composition concentrate comprising
an amount of a hypochlorite salt, wherein the amount of the hypochlorite salt is about 0.0001% to about 1.5% by weight of the concentrate;
an amount of a humectant, wherein the amount of the humectant is about 15% to about 35% by weight of the concentrate;
an amount of water, wherein the amount of water is about 60% to about 80% by weight of the concentrate; and
an amount of a stabilizer, wherein the amount of the stabilizer is about 0.5% to about 5.0% by weight of the concentrate.

In one embodiment, the invention relates to a bleach composition concentrate consisting essentially of
an amount of a hypochlorite salt, wherein the amount of the hypochlorite salt is about 0.0001% to about 1.5% by weight of the concentrate;
an amount of a humectant, wherein the amount of the humectant is about 15% to about 35% by weight of the concentrate;
an amount of water, wherein the amount of water is about 60% to about 80% by weight of the concentrate; and
an amount of a stabilizer, wherein the amount of the stabilizer is about 0.5% to about 5.0% by weight of the concentrate.

In one embodiment, the invention relates to a bleach composition concentrate consisting of
an amount of a hypochlorite salt, wherein the amount of the hypochlorite salt is about 0.0001% to about 1.5% by weight of the concentrate;
an amount of a humectant, wherein the amount of the humectant is about 15% to about 35% by weight of the concentrate;
an amount of water, wherein the amount of water is about 60% to about 80% by weight of the concentrate; and
an amount of a stabilizer, wherein the amount of the stabilizer is about 0.5% to about 5.0% by weight of the concentrate.

In one embodiment, the invention relates to a bleach composition concentrate comprising
an amount of a hypochlorite salt, wherein the amount of the hypochlorite salt is about 0.0001% to about 1.5% by weight of the concentrate;
an amount of a viscosity modifier, wherein the amount of the viscosity modifier is about 0.1% to about 6% by weight of the concentrate;
an amount of a surfactant, wherein the amount of the surfactant is about 0.01% to about 1% by weight of the concentrate;
an amount of water, wherein the amount of water is about 80% to about 99% by weight of the concentrate;
an amount of a stabilizer, wherein the amount of the stabilizer is about 0.01% to about 1.0% by weight of the concentrate; and
an amount of a pH modifier, wherein the amount of the pH modifier is from about 0.01% to about 0.06% by weight of the concentrate.

In one embodiment, the invention relates to a bleach composition concentrate consisting essentially of
an amount of a hypochlorite salt, wherein the amount of the hypochlorite salt is about 0.0001% to about 1.5% by weight of the concentrate;
an amount of a viscosity modifier, wherein the amount of the viscosity modifier is about 0.1% to about 6% by weight of the concentrate;
an amount of a surfactant, wherein the amount of the surfactant is about 0.01% to about 1% by weight of the concentrate;
an amount of water, wherein the amount of water is about 80% to about 99% by weight of the concentrate;
an amount of a stabilizer, wherein the amount of the stabilizer is about 0.01% to about 1.0% by weight of the concentrate; and
an amount of a pH modifier, wherein the amount of the pH modifier is from about 0.01% to about 0.06% by weight of the concentrate.

In one embodiment, the invention relates to a bleach composition concentrate consisting of
an amount of a hypochlorite salt, wherein the amount of the hypochlorite salt is about 0.0001% to about 1.5% by weight of the concentrate;
an amount of a viscosity modifier, wherein the amount of the viscosity modifier is about 0.1% to about 6% by weight of the concentrate;
an amount of a surfactant, wherein the amount of the surfactant is about 0.01% to about 1% by weight of the concentrate;
an amount of water, wherein the amount of water is about 80% to about 99% by weight of the concentrate;
an amount of a stabilizer, wherein the amount of the stabilizer is about 0.01% to about 1.0% by weight of the concentrate; and
an amount of a pH modifier, wherein the amount of the pH modifier is from about 0.01% to about 0.06% by weight of the concentrate.

In one embodiment, the invention relates to a bleach formulation, wherein the bleach formulation comprises any one of the above-mentioned bleach composition concentrates; and a propellant. In one embodiment, the invention relates to any one of the above-mentioned bleach formulations, further comprising a hydrofluoroalkane propellant.

In one embodiment, the invention relates to any one of the above-mentioned bleach formulations, wherein the hydrofluoroalkane propellant is 1,1,1,2-tetrafluoroethane, 1,1,1,2,3,3,3-heptafluoropropane, or a mixture thereof.

In one embodiment, the invention relates to any one of the above-mentioned bleach formulations, wherein the hydrofluoroalkane propellant is 1,1,1,2-tetrafluoroethane.

In one embodiment, the invention relates to any one of the above-mentioned bleach compositions (either a bleach composition concentrate or a bleach formulation), wherein the hypochlorite salt is present in an amount from about 0.0001% to about 1.5% by weight of the concentrate. In one embodiment, the invention relates to any one of the above-mentioned bleach compositions, wherein the hypochlorite salt is present in an amount from about 0.001% to about 0.8% by weight of the concentrate. In one embodiment, the invention relates to any one of the above-mentioned bleach compositions, wherein the hypochlorite salt is present in an amount from about 0.01% to about 0.5% by weight of the concentrate. In one embodiment, the invention relates to any one of the above-mentioned bleach compositions, wherein the hypochlorite salt is present in an amount from about 0.1% to about 0.25% by weight of the concentrate. In one embodiment, the invention relates to any one of the above-mentioned compositions, wherein the hypochlorite salt is present in about 0.0001%, about 0.0005%, about 0.001%, about 0.005%, about 0.01%, about 0.05%, about 0.1%, about 0.2%, about 0.3%, about 0.4%, about 0.5%, about 0.6%, about 0.7%, about 0.8%, about 0.9%, or about 1.5% by weight of the concentrate.

In one embodiment, the invention relates to any one of the above-mentioned bleach compositions, wherein the hypochlorite salt is sodium hypochlorite, potassium hypochlorite, calcium hypochlorite, magnesium hypochlorite, lithium hypochlorite, or copper(I) or copper(II) hypochlorite. In one embodiment, the invention relates to any one of the above-mentioned bleach compositions, wherein the hypochlorite salt is sodium hypochlorite or calcium hypochlorite.

In one embodiment, the invention relates to any one of the above-mentioned bleach compositions, wherein water is present in an amount from about 60% to about 80% by weight of the concentrate. In one embodiment, the invention relates to any one of the above-mentioned bleach compositions, wherein water is present in an amount from about 65% to about 75% by weight of the concentrate. In one embodiment, the invention relates to any one of the above-mentioned bleach compositions, wherein water is present in an amount from about 68% to about 72% by weight of the concentrate. In one embodiment, the invention relates to any one of the above-mentioned bleach compositions, wherein water is present in about 60%, about 65%, about 70%, about 75%, or about 80% by weight of the concentrate. In one embodiment, the invention relates to any one of the above-mentioned bleach compositions, wherein water is present in an amount from about 80% to about 99% by weight of the concentrate. In one embodiment, the invention relates to any one of the above-mentioned bleach compositions, wherein water is present in about 80%, about 85%, about 90%, about 91%, about 92%, about 93%, about 94%, about 95%, about 96%, about 97%, about 98%, or about 99% by weight of the concentrate.

In one embodiment, the invention relates to any one of the above-mentioned bleach compositions, wherein the stabilizer is selected from the group consisting of DL-alpha tocopheryl acetate, imidazolidinyl urea, diazolidinyl urea, phenoxyethanol, sodium methyl paraben, methylparaben, ethylparaben, propylparaben, potassium sorbate, sodium benzoate, sodium chloride, sorbic acid, benzoic acid, formaldehyde, citric acid, sodium citrate, chlorine dioxide, benzalkonium chloride, benzethonium chloride, cetrimide, dequalinium chloride, cetylpyridinium chloride, phenylmercuric nitrate, phenylmercuric acetate, thimerosal, chlorobutanol, dichlorobenzyl alcohol, phenylethyl alcohol, benzyl alcohol, ascorbic acid, sodium bisulfite, butylated hydroxytoluene, butylated hydroxyanisole, α-tocopherol, sodium ascorbate, ascorbyl palmitate, propyl gallate, disodium EDTA, and mixtures thereof.

In one embodiment, the invention relates to any one of the above-mentioned bleach compositions, wherein the stabilizer is DL-alpha tocopheryl acetate, methylparaben, sodium chloride, propylparaben, disodium EDTA, or a mixture thereof. In one embodiment, the invention relates to any one of the above-mentioned compositions, wherein the stabilizer is sodium chloride.

In one embodiment, the invention relates to any one of the above-mentioned bleach compositions, wherein the stabilizer is present in an amount from about 0.001% to about 1.0% by weight of the concentrate. In one embodiment, the invention relates to any one of the above-mentioned bleach compositions, wherein the stabilizer is present in an amount from about 0.8% to about 4.0% by weight of the concentrate. In one embodiment, the invention relates to any one of the above-mentioned bleach compositions, wherein the stabilizer is present in an amount from about 1.0% to about 3.0% by weight of the concentrate. In one embodiment, the invention relates to any one of the above-mentioned bleach compositions, wherein the stabilizer is present in about 0.001%, about 0.002%, about 0.003%, about 0.004%, about 0.005%, about 0.006%, about 0.007%, about 0.008%, about 0.009%, about 0.01%, about 0.05%, about 0.1%, about 0.5%, about 0.8%, about 1.0%, about 1.5%, about 2.0%, about 2.5%, about 3.0%, about 3.5%, about 4.0%, about 4.5%, or about 5.0% by weight of the concentrate.

In one embodiment, the invention relates to any one of the above-mentioned bleach compositions, wherein the humectant is selected from the group consisting of 2-ethylhexyl palmitate, sodium hyaluronate, glycerol, PPG-15 stearyl ether, lanolin alcohol, lanolin, cholesterol, petrolatum, isostearyl neopentanoate, octyl stearate, mineral oil, isocetyl stearate, myristyl myristate, octyl dodecanol, dimethicone, phenyl trimethicone, cyclomethicone, C₁₂-C₁₅ alkyl benzoates, dimethiconol, propylene glycol, lactic acid, butylene glycol, sodium PCA, carbowax 200, carbowax 400, carbowax 800, and mixtures thereof.

In one embodiment, the invention relates to any one of the above-mentioned bleach compositions, wherein the humectant is 2-ethylhexyl palmitate, sodium hyaluronate, glycerol, petrolatum, dimethicone, propylene glycol, or a mixture thereof.

In one embodiment, the invention relates to any one of the above-mentioned bleach compositions, wherein the humectant is present in an amount from about 0.5% to about 32% by weight of the concentrate. In one embodiment, the invention relates to any one of the above-mentioned bleach compositions, wherein the humectant is present in an amount from about 0.8% to about 30% by weight of the concentrate. In one embodiment, the invention relates to any one of the above-mentioned bleach compositions, wherein the humectant is present in an amount from about 22% to about 28% by weight of the concentrate. In one embodiment, the invention relates to any one of the above-mentioned bleach compositions, wherein the humectant is present in about 1%, about 2%, about 3%, about 4%, about 5%, about 10%, about 15%, about 16%, about 17%, about 18%, about 19%, about 20%, about 22%, about 25%, about 28%, about 30%, about 32%, or about 35% by weight of the concentrate. In one embodiment, the humectant is present in an amount from about 5% to about 15% by weight of the concentrate.

In one embodiment, the invention relates to any one of the above-mentioned bleach compositions, wherein the concentrate further comprises an emulsifier or a surfactant.

In one embodiment, the invention relates to any one of the above-mentioned bleach compositions, wherein the emulsifier or the surfactant is selected from the group consisting of dicetyl phosphate, polyethylene glycol hexadecyl ether phosphate, polyoxyethylene monooctadecyl ether, polysorbate 20, polysorbate 40, polysorbate 60, polysorbate 80, steareth-10, sodium dodecyl sulfate, lauryl dimethyl amine oxide, cetyltrimethylammonium bromide (CTAB), polyoxyethylene sorbitan, octoxynol, N,N-dimethyldodecylamine-N-oxide, hexadecyltrimethylammonium bromide (HTAB), polyoxyl 10 lauryl ether, sodium deoxycholate, sodium cholate, polyoxyl castor oil, nonylphenol ethoxylate, cyclodextrins, lecithin, dimethicone copolyol, lauramide DEA, cocamide DEA, cocamide MEA, oleyl betaine, cocamidopropyl betaine, cocamidopropyl phosphatidyl PG-dimonium chloride, methylbenzethonium chloride, behentrimonium methosulfate-cetearyl alcohol, emulsifying wax, polyoxyethylene oleyl ether, PEG-40 stearate, cetostearyl alcohol, ceteareth-12, ceteareth-20, ceteareth-30, ceteareth alcohol, glyceryl stearate, PEG-100 stearate, glyceryl stearate, PEG-100 stearate, steareth-2, steareth-20, stearamidopropyl dimethylamine, behentrimonium methosulfate, alkyl polyglucoside, and mixtures thereof.

In one embodiment, the invention relates to any one of the above-mentioned bleach compositions, wherein the emulsifier or the surfactant is dicetyl phosphate, polyethylene glycol hexadecyl ether phosphate, polyoxyethylene monooctadecyl ether, cetostearyl alcohol, alkyl polyglucoside, or a mixture thereof.

In one embodiment, the invention relates to any one of the above-mentioned bleach compositions, wherein the emulsifier or the surfactant is present in an amount from about 0.01% to about 1% by weight of the concentrate. In one embodiment, the invention relates to any one of the above-mentioned bleach compositions, wherein the emulsifier or the surfactant is present in an amount from about 2% to about 10% by weight of the concentrate. In one embodiment, the invention relates to any one of the above-mentioned bleach compositions, wherein the emulsifier or the surfactant is present in an amount from about 3% to about 9% by weight of the concentrate. In one embodiment, the invention relates to any one of the above-mentioned bleach compositions, wherein the emulsifier or the surfactant is present in an amount from about 4% to about 8% by weight of the concentrate. In one embodiment, the invention relates to any one of the above-mentioned bleach compositions, wherein the emulsifier or the surfactant is present in an amount from about 3% to about 7% by weight of the concentrate. In one embodiment, the invention relates to any one of the above-mentioned bleach compositions, wherein the emulsifier or the surfactant is present in about 4%, about 5%, about 6%, about 7%, or about 8% by weight of the concentrate.

In one embodiment, the invention relates to any one of the above-mentioned bleach compositions, wherein the concentrate further comprises a pH adjusting agent. In one embodiment, the invention relates to any one of the above-mentioned compositions, wherein the pH adjusting agent is sodium hydroxide. In various embodiments, the invention relates to any one of the above-mentioned bleach compositions, wherein the pH adjusting agent is monobasic sodium phosphate, dibasic sodium phosphate, or a combination of monobasic sodium phosphate and dibasic sodium phosphate.

In one embodiment, the invention relates to any one of the above-mentioned bleach compositions, wherein the concentrate further comprises a natural extract. In one embodiment, the natural extract is a fat or glycidic oil from Theobroma grandiflorum.

In one embodiment, the invention relates to any one of the above-mentioned bleach compositions, wherein the composition is colorless. In one embodiment, the invention relates to any one of the above-mentioned bleach compositions, wherein the composition is off-white.

In one embodiment, the invention relates to any one of the above-mentioned bleach compositions, wherein the composition is low odor. In one embodiment, the invention relates to any one of the above-mentioned bleach compositions, wherein the composition is fragrance-free.

In one embodiment, the invention relates to any one of the above-mentioned bleach compositions, wherein the composition has a pH from about 5.5 to about 9. In one embodiment, the invention relates to any one of the above-mentioned bleach compositions, wherein the composition has a pH from about 5.5 to about 8. In one embodiment, the invention relates to any one of the above-mentioned bleach compositions, wherein the composition has a pH of about 5.5, about 6.0, about 6.5, about 7.0, about 7.5, or about 8.0.

In one embodiment, the invention relates to any one of the above-mentioned bleach compositions, wherein the concentrate has a pH from about 5.5 to about 9. In one embodiment, the invention relates to any one of the above-mentioned bleach compositions, wherein the concentrate has a pH from about 5.5 to about 8. In one embodiment, the invention relates to any one of the above-mentioned bleach compositions, wherein the concentrate has a pH of about 5.5, about 6.0, about 6.5, about 7.0, about 7.5, or about 8.0.

In one embodiment, the invention relates to any one of the above-mentioned bleach formulations, wherein the concentrate is in an aerosol container.

In one embodiment, the invention relates to any one of the above-mentioned bleach formulations, wherein the concentrate is in an aerosol container, thereby forming a headspace of the aerosol container; and the headspace of the aerosol container is substantially free of oxygen.

In one embodiment, the invention relates to any one of the above-mentioned bleach formulations, wherein the concentrate is in an aerosol container, thereby forming a headspace of the aerosol container; and the headspace of the aerosol container consists essentially of argon.

In one embodiment, the invention relates to any one of the above-mentioned bleach formulations, wherein when the aerosol container is actuated, the formulation is expelled as a foam.

In one embodiment, the invention relates to any one of the above-mentioned bleach formulations, wherein the concentrate is in an aerosol container. In one embodiment, the invention relates to any one of the above-mentioned bleach formulations, wherein the formulation is about 4% to about 50% propellant, by weight of the formulation. In one embodiment, the invention relates to any one of the above-mentioned bleach formulations, wherein the formulation is about 5% to about 40% propellant, by weight of the formulation. In one embodiment, the invention relates to any one of the above-mentioned bleach formulations, wherein the formulation is about 6% to about 30% propellant, by weight of the formulation. In one embodiment, the invention relates to any one of the above-mentioned bleach formulations, wherein the formulation is about 6% to about 18% propellant, by weight of the formulation. In one embodiment, the invention relates to any one of the above-mentioned bleach formulations, wherein the formulation is about 6%, about 7%, about 8%, about 9%, about 10%, about 11%, about 12%, about 13%, about 14%, about 15%, about 20%, about 25%, or about 30% propellant, by weight of the formulation. In one embodiment, the invention relates to any one of the above-mentioned bleach formulations, wherein the formulation is about 12% propellant, by weight of the formulation. In one embodiment, the invention relates to any one of the above-mentioned bleach formulations, wherein about 6%, about 7%, about 8%, about 9%, about 10%, about 11%, about 12%, about 13%, about 14%, about 15%, about 20%, about 25%, or about 30% propellant, by weight of the formulation, is required to deliver the concentrate as a stable foam.

In one embodiment, the topical bleach compositions are commercially available products such as Microcyn®, produced by Oculus Innovative Sciences (Electrolyzed Water (96.524%), Sodium Magnesium Fluorosilicate (3.000%), Sodium Phosphate (0.400%), Sodium Chloride (0.066%), Hypochlorous Acid (0.008%), and Sodium Hypochlorite (0.002%)), or Anasept®, produced by Anacapa Technologies (sodium hypochlorite, sodium chloride, sodium magnesium silicate, and water).

In one embodiment, the invention relates to a bleach composition in the form of a gel. In one embodiment, the bleach composition comprises: (a) a sodium hypochlorite solution that is not prepared by the partial electrolysis of a sodium chloride solution; (b) at least one viscosity-enhancing agent chosen from magnesium aluminum silicates, smectite clays, allophone, kaolinite, nacarite, halloysites, sodium montmorillonite, calcium montmorillonite, sauconite, vermiculite, nontronite, saponite, hectorite, bentonite, attapulgite, sepiolite, palygorskite, and mixtures thereof; and (c) at least one electrolyte chosen from USP sodium chloride, USP citric acid, and NF hydrochloric acid; wherein the gel composition has a pH value ranging from about 5.5 to about 9; the sodium hypochlorite is present in the gel composition in an amount ranging from about 0.0125% to about 1% by weight with respect to the total weight of the gel composition; the at least one viscosity-enhancing agent is present in the composition in an amount ranging from about 0.1 to about 10% by weight relative to the total weight of the composition; and the at least one electrolyte is present in the composition in an amount ranging from about 0.01 to about 10% by weight relative to the total weight of the composition. In one embodiment, the bleach composition comprises: (a) a sodium hypochlorite solution that is not prepared by the partial electrolysis of a sodium chloride solution; (b) at least one viscosity-enhancing agent chosen from magnesium aluminum silicates, smectite clays, allophone, kaolinite, nacarite, halloysites, sodium montmorillonite, calcium montmorillonite, sauconite, vermiculite, nontronite, saponite, hectorite, bentonite, attapulgite, sepiolite, palygorskite, and mixtures thereof; and (c) at least one electrolyte chosen from USP sodium chloride, USP citric acid, and NF hydrochloric acid; wherein the gel composition has a pH value ranging from about 5.5 to about 9; the sodium hypochlorite is present in the gel composition in an amount ranging from about 0.0125% to about 1% by weight with respect to the total weight of the gel composition; the at least one viscosity-enhancing agent is present in the composition in an amount ranging from about 0.1 to about 10% by weight relative to the total weight of the composition; and the at least one electrolyte is present in the composition in an amount ranging from about 0.01 to about 10% by weight relative to the total weight of the composition.

Exemplary Properties of Bleach Compositions

In one embodiment, the invention relates to any one of the above-mentioned bleach compositions (for example, either a bleach composition concentrate or a bleach formulation), wherein the composition is in the form of a foam.

In one embodiment, the invention relates to any one of the above-mentioned bleach formulations, wherein the formulation produces a foam.

In one embodiment, the invention relates to any one of the above-mentioned bleach formulations, wherein the foam is produced by actuation of an aerosol container comprising the formulation.

In one embodiment, the invention relates to any one of the above-mentioned bleach compositions, wherein the foam is non-irritating when applied to the skin of a subject.

In one embodiment, the invention relates to any one of the above-mentioned bleach compositions, wherein the composition does not comprise methanol, ethanol, propanols, or butanols.

In one embodiment, the invention relates to any one of the above-mentioned bleach compositions, wherein the composition does not comprise methane, ethane, propane, butane, pentane, or hexane.

In one embodiment, the invention relates to any one of the above-mentioned bleach compositions, wherein the composition is non-irritating when applied to the skin.

In one embodiment, the invention relates to any one of the above-mentioned bleach compositions, wherein the composition is non-sterile. In one embodiment, the invention relates to any one of the above-mentioned bleach compositions, wherein the composition is sterile.

Exemplary Barrier Repair Compositions

In one embodiment, the barrier repair composition is a drug-free topical cream, lotion, or aerosol foam.

In certain embodiments, the invention relates to a barrier repair emulsion, comprising:
an oil phase, wherein the oil phase comprises an emulsifier or surfactant, a first moisturizer or first emollient, and a first antioxidant or first preservative; and
an aqueous phase, wherein the aqueous phase comprises a vehicle, a second moisturizer or second emollient, a second antioxidant or second preservative, and a pH adjuster.

In certain embodiments, the invention relates to a barrier repair emulsion, consisting essentially of:
an oil phase, wherein the oil phase consists essentially of an emulsifier or surfactant, a first moisturizer or first emollient, and a first antioxidant or first preservative; and
an aqueous phase, wherein the aqueous phase consists essentially of a vehicle, a second moisturizer or second emollient, a second antioxidant or second preservative, and a pH adjuster.

In certain embodiments, the invention relates to a barrier repair emulsion, consisting of:
an oil phase, wherein the oil phase consists of an emulsifier or surfactant, a first moisturizer or first emollient, and a first antioxidant or first preservative; and
an aqueous phase, wherein the aqueous phase consists of a vehicle, a second moisturizer or second emollient, a second antioxidant or second preservative, and a pH adjuster.

In certain embodiments, the invention relates to a barrier repair emulsion, comprising:
cetearyl alcohol, from about 1% to about 4% by weight of the emulsion;
dicetyl phosphate, from about 1% to about 4% by weight of the emulsion;
ceteareth-10 phosphate, from about 0.5% to about 2% by weight of the emulsion;
steareth-10, from about 0.4% to about 1.3% by weight of the emulsion;
Theobroma grandiflorum seed butter, from about 1% to about 3% by weight of the emulsion;
white petrolatum, from about 0.5% to about 1.5% by weight of the emulsion;
dimethicone, from about 0.5% to about 1.5% by weight of the emulsion;
2-ethylhexyl palmitate, from about 3% to about 9% by weight of the emulsion;
hydroxypropyl bispalmitamide MEA, from about 0.2% to about 0.8% by weight of the emulsion;
tocopheryl acetate, from about 0.2% to about 0.8% by weight of the emulsion;
water, from about 55% to about 85% by weight of the emulsion;
propylene glycol, from about 1.2% to about 3.8% by weight of the emulsion;
glycerol, from about 4% to about 11% by weight of the emulsion;
sodium hyaluronate, from about 0.05% to about 0.2% by weight of the emulsion;
methylparaben, from about 0.1% to about 0.5% by weight of the emulsion;
propylparaben, from about 0.05% to about 0.2% by weight of the emulsion;
disodium EDTA, from about 0.05% to about 0.2% by weight of the emulsion; and
sodium hydroxide.

In certain embodiments, the invention relates to a barrier repair emulsion, consisting essentially of:
cetearyl alcohol, from about 1% to about 4% by weight of the emulsion;
dicetyl phosphate, from about 1% to about 4% by weight of the emulsion;
ceteareth-10 phosphate, from about 0.5% to about 2% by weight of the emulsion;
steareth-10, from about 0.4% to about 1.3% by weight of the emulsion;
Theobroma grandiflorum seed butter, from about 1% to about 3% by weight of the emulsion;
white petrolatum, from about 0.5% to about 1.5% by weight of the emulsion;
dimethicone, from about 0.5% to about 1.5% by weight of the emulsion;
2-ethylhexyl palmitate, from about 3% to about 9% by weight of the emulsion;
hydroxypropyl bispalmitamide MEA, from about 0.2% to about 0.8% by weight of the emulsion;
tocopheryl acetate, from about 0.2% to about 0.8% by weight of the emulsion;
water, from about 55% to about 85% by weight of the emulsion;
propylene glycol, from about 1.2% to about 3.8% by weight of the emulsion;
glycerol, from about 4% to about 11% by weight of the emulsion;
sodium hyaluronate, from about 0.05% to about 0.2% by weight of the emulsion;
methylparaben, from about 0.1% to about 0.5% by weight of the emulsion;
propylparaben, from about 0.05% to about 0.2% by weight of the emulsion;
disodium EDTA, from about 0.05% to about 0.2% by weight of the emulsion; and
sodium hydroxide.

In certain embodiments, the invention relates to a barrier repair emulsion, consisting of:
cetearyl alcohol, from about 1% to about 4% by weight of the emulsion;
dicetyl phosphate, from about 1% to about 4% by weight of the emulsion;
ceteareth-10 phosphate, from about 0.5% to about 2% by weight of the emulsion;
steareth-10, from about 0.4% to about 1.3% by weight of the emulsion;
Theobroma grandiflorum seed butter, from about 1% to about 3% by weight of the emulsion;
white petrolatum, from about 0.5% to about 1.5% by weight of the emulsion;
dimethicone, from about 0.5% to about 1.5% by weight of the emulsion;
2-ethylhexyl palmitate, from about 3% to about 9% by weight of the emulsion;
hydroxypropyl bispalmitamide MEA, from about 0.2% to about 0.8% by weight of the emulsion;
tocopheryl acetate, from about 0.2% to about 0.8% by weight of the emulsion;
water, from about 55% to about 85% by weight of the emulsion;
propylene glycol, from about 1.2% to about 3.8% by weight of the emulsion;
glycerol, from about 4% to about 11% by weight of the emulsion;
sodium hyaluronate, from about 0.05% to about 0.2% by weight of the emulsion;
methylparaben, from about 0.1% to about 0.5% by weight of the emulsion;
propylparaben, from about 0.05% to about 0.2% by weight of the emulsion;
disodium EDTA, from about 0.05% to about 0.2% by weight of the emulsion; and
sodium hydroxide.

In certain embodiments, the invention relates to a barrier repair emulsion, comprising:
cetearyl alcohol, in about 2.4% by weight of the emulsion; dicetyl phosphate, in about 2.4% by weight of the emulsion;
ceteareth-10 phosphate, in about 1.2% by weight of the emulsion;
steareth-10, in about 0.9% by weight of the emulsion;
Theobroma grandiflorum seed butter, in about 2% by weight of the emulsion;
white petrolatum, in about 1% by weight of the emulsion;
dimethicone, in about 1% by weight of the emulsion;
2-ethylhexyl palmitate, in about 6% by weight of the emulsion;
hydroxypropyl bispalmitamide MEA, in about 0.5% by weight of the emulsion;
tocopheryl acetate, in about 0.5% by weight of the emulsion;
water, in about 71.5% by weight of the emulsion;
propylene glycol, in about 2.5% by weight of the emulsion;
glycerol, in about 7.5% by weight of the emulsion;
sodium hyaluronate, in about 0.1% by weight of the emulsion;
methylparaben, in about 0.3% by weight of the emulsion;
propylparaben, in about 0.1% by weight of the emulsion;
disodium EDTA, in about 0.1% by weight of the emulsion; and
sodium hydroxide.

In certain embodiments, the invention relates to a barrier repair emulsion, consisting essentially of:
cetearyl alcohol, in about 2.4% by weight of the emulsion; dicetyl phosphate, in about 2.4% by weight of the emulsion;
ceteareth-10 phosphate, in about 1.2% by weight of the emulsion;
steareth-10, in about 0.9% by weight of the emulsion;
Theobroma grandiflorum seed butter, in about 2% by weight of the emulsion;
white petrolatum, in about 1% by weight of the emulsion;
dimethicone, in about 1% by weight of the emulsion;
2-ethylhexyl palmitate, in about 6% by weight of the emulsion;
hydroxypropyl bispalmitamide MEA, in about 0.5% by weight of the emulsion;
tocopheryl acetate, in about 0.5% by weight of the emulsion;
water, in about 71.5% by weight of the emulsion;
propylene glycol, in about 2.5% by weight of the emulsion;
glycerol, in about 7.5% by weight of the emulsion;
sodium hyaluronate, in about 0.1% by weight of the emulsion;
methylparaben, in about 0.3% by weight of the emulsion;
propylparaben, in about 0.1% by weight of the emulsion;
disodium EDTA, in about 0.1% by weight of the emulsion; and
sodium hydroxide.

In certain embodiments, the invention relates to a barrier repair emulsion, consisting of:
cetearyl alcohol, in about 2.4% by weight of the emulsion;
dicetyl phosphate, in about 2.4% by weight of the emulsion;
ceteareth-10 phosphate, in about 1.2% by weight of the emulsion;
steareth-10, in about 0.9% by weight of the emulsion;
Theobroma grandiflorum seed butter, in about 2% by weight of the emulsion;
white petrolatum, in about 1% by weight of the emulsion;
dimethicone, in about 1% by weight of the emulsion;
2-ethylhexyl palmitate, in about 6% by weight of the emulsion;
hydroxypropyl bispalmitamide MEA, in about 0.5% by weight of the emulsion;
tocopheryl acetate, in about 0.5% by weight of the emulsion;
water, in about 71.5% by weight of the emulsion;
propylene glycol, in about 2.5% by weight of the emulsion;
glycerol, in about 7.5% by weight of the emulsion;
sodium hyaluronate, in about 0.1% by weight of the emulsion;
methylparaben, in about 0.3% by weight of the emulsion;
propylparaben, in about 0.1% by weight of the emulsion;
disodium EDTA, in about 0.1% by weight of the emulsion; and
sodium hydroxide.

In certain embodiments, the invention relates to a barrier repair formulation, comprising:

an emulsion, wherein the emulsion comprises:
an oil phase, wherein the oil phase comprises an emulsifier or surfactant, a first moisturizer or first emollient, and a first antioxidant or first preservative; and
an aqueous phase, wherein the aqueous phase comprises a vehicle, a second moisturizer or second emollient, a second antioxidant or second preservative, and a pH adjuster;

a propellant; and

a purge gas.

In certain embodiments, the invention relates to a barrier repair formulation, consisting essentially of:

an emulsion, wherein the emulsion consists essentially of:
an oil phase, wherein the oil phase consists essentially of an emulsifier or surfactant, a first moisturizer or first emollient, and a first antioxidant or first preservative; and
an aqueous phase, wherein the aqueous phase consists essentially of a vehicle, a second moisturizer or second emollient, a second antioxidant or second preservative, and a pH adjuster;

a propellant; and

a purge gas.

In certain embodiments, the invention relates to a barrier repair formulation, consisting of:

an emulsion, wherein the emulsion consists of:
an oil phase, wherein the oil phase consists of an emulsifier or surfactant, a first moisturizer or first emollient, and a first antioxidant or first preservative; and
an aqueous phase, wherein the aqueous phase consists of a vehicle, a second moisturizer or second emollient, a second antioxidant or second preservative, and a pH adjuster;

a propellant; and

a purge gas.

In certain embodiments, the invention relates to a barrier repair formulation, comprising:

an emulsion, wherein the emulsion comprises:
cetearyl alcohol, from about 1% to about 4% by weight of the emulsion;
dicetyl phosphate, from about 1% to about 4% by weight of the emulsion;
ceteareth-10 phosphate, from about 0.5% to about 2% by weight of the emulsion;
steareth-10, from about 0.4% to about 1.3% by weight of the emulsion;
Theobroma grandiflorum seed butter, from about 1% to about 3% by weight of the emulsion;
white petrolatum, from about 0.5% to about 1.5% by weight of the emulsion;
dimethicone, from about 0.5% to about 1.5% by weight of the emulsion;
2-ethylhexyl palmitate, from about 3% to about 9% by weight of the emulsion;
hydroxypropyl bispalmitamide MEA, from about 0.2% to about 0.8% by weight of the emulsion;
tocopheryl acetate, from about 0.2% to about 0.8% by weight of the emulsion;
water, from about 55% to about 85% by weight of the emulsion;
propylene glycol, from about 1.2% to about 3.8% by weight of the emulsion;
glycerol, from about 4% to about 11% by weight of the emulsion;
sodium hyaluronate, from about 0.05% to about 0.2% by weight of the emulsion;
methylparaben, from about 0.1% to about 0.5% by weight of the emulsion;
propylparaben, from about 0.05% to about 0.2% by weight of the emulsion;
disodium EDTA, from about 0.05% to about 0.2% by weight of the emulsion; and
sodium hydroxide;

a propellant; and

a purge gas.

In certain embodiments, the invention relates to a barrier repair formulation, consisting essentially of:

an emulsion, wherein the emulsion consists essentially of:
cetearyl alcohol, from about 1% to about 4% by weight of the emulsion;
dicetyl phosphate, from about 1% to about 4% by weight of the emulsion;
ceteareth-10 phosphate, from about 0.5% to about 2% by weight of the emulsion;
steareth-10, from about 0.4% to about 1.3% by weight of the emulsion;
Theobroma grandiflorum seed butter, from about 1% to about 3% by weight of the emulsion;
white petrolatum, from about 0.5% to about 1.5% by weight of the emulsion;
dimethicone, from about 0.5% to about 1.5% by weight of the emulsion;
2-ethylhexyl palmitate, from about 3% to about 9% by weight of the emulsion;
hydroxypropyl bispalmitamide MEA, from about 0.2% to about 0.8% by weight of the emulsion;
tocopheryl acetate, from about 0.2% to about 0.8% by weight of the emulsion;
water, from about 55% to about 85% by weight of the emulsion;
propylene glycol, from about 1.2% to about 3.8% by weight of the emulsion;
glycerol, from about 4% to about 11% by weight of the emulsion;
sodium hyaluronate, from about 0.05% to about 0.2% by weight of the emulsion;
methylparaben, from about 0.1% to about 0.5% by weight of the emulsion;
propylparaben, from about 0.05% to about 0.2% by weight of the emulsion;
disodium EDTA, from about 0.05% to about 0.2% by weight of the emulsion; and
sodium hydroxide;

a propellant; and

a purge gas.

In certain embodiments, the invention relates to a barrier repair formulation, consisting of:

an emulsion, wherein the emulsion consists of:
cetearyl alcohol, from about 1% to about 4% by weight of the emulsion;
dicetyl phosphate, from about 1% to about 4% by weight of the emulsion;
ceteareth-10 phosphate, from about 0.5% to about 2% by weight of the emulsion;
steareth-10, from about 0.4% to about 1.3% by weight of the emulsion;
Theobroma grandiflorum seed butter, from about 1% to about 3% by weight of the emulsion;
white petrolatum, from about 0.5% to about 1.5% by weight of the emulsion;
dimethicone, from about 0.5% to about 1.5% by weight of the emulsion;
2-ethylhexyl palmitate, from about 3% to about 9% by weight of the emulsion;
hydroxypropyl bispalmitamide MEA, from about 0.2% to about 0.8% by weight of the emulsion;
tocopheryl acetate, from about 0.2% to about 0.8% by weight of the emulsion;
water, from about 55% to about 85% by weight of the emulsion;
propylene glycol, from about 1.2% to about 3.8% by weight of the emulsion;
glycerol, from about 4% to about 11% by weight of the emulsion;
sodium hyaluronate, from about 0.05% to about 0.2% by weight of the emulsion;
methylparaben, from about 0.1% to about 0.5% by weight of the emulsion;
propylparaben, from about 0.05% to about 0.2% by weight of the emulsion;
disodium EDTA, from about 0.05% to about 0.2% by weight of the emulsion; and
sodium hydroxide;

a propellant; and

a purge gas.

In certain embodiments, the invention relates to a barrier repair formulation, comprising:

an emulsion, wherein the emulsion comprises:
cetearyl alcohol, in about 2.4% by weight of the emulsion; dicetyl phosphate, in about 2.4% by weight of the emulsion;
ceteareth-10 phosphate, in about 1.2% by weight of the emulsion;
steareth-10, in about 0.9% by weight of the emulsion;
Theobroma grandiflorum seed butter, in about 2% by weight of the emulsion;
white petrolatum, in about 1% by weight of the emulsion;
dimethicone, in about 1% by weight of the emulsion;
2-ethylhexyl palmitate, in about 6% by weight of the emulsion;
hydroxypropyl bispalmitamide MEA, in about 0.5% by weight of the emulsion;
tocopheryl acetate, in about 0.5% by weight of the emulsion;
water, in about 71.5% by weight of the emulsion;
propylene glycol, in about 2.5% by weight of the emulsion;
glycerol, in about 7.5% by weight of the emulsion;
sodium hyaluronate, in about 0.1% by weight of the emulsion;
methylparaben, in about 0.3% by weight of the emulsion;
propylparaben, in about 0.1% by weight of the emulsion;
disodium EDTA, in about 0.1% by weight of the emulsion; and
sodium hydroxide;

a propellant; and

a purge gas.

In certain embodiments, the invention relates to a barrier repair formulation, consisting essentially of:

an emulsion, wherein the emulsion consists essentially of:
cetearyl alcohol, in about 2.4% by weight of the emulsion;
dicetyl phosphate, in about 2.4% by weight of the emulsion;
ceteareth-10 phosphate, in about 1.2% by weight of the emulsion;
steareth-10, in about 0.9% by weight of the emulsion;
Theobroma grandiflorum seed butter, in about 2% by weight of the emulsion;
white petrolatum, in about 1% by weight of the emulsion;
dimethicone, in about 1% by weight of the emulsion;
2-ethylhexyl palmitate, in about 6% by weight of the emulsion;
hydroxypropyl bispalmitamide MEA, in about 0.5% by weight of the emulsion;
tocopheryl acetate, in about 0.5% by weight of the emulsion;
water, in about 71.5% by weight of the emulsion;
propylene glycol, in about 2.5% by weight of the emulsion;
glycerol, in about 7.5% by weight of the emulsion;
sodium hyaluronate, in about 0.1% by weight of the emulsion;
methylparaben, in about 0.3% by weight of the emulsion;
propylparaben, in about 0.1% by weight of the emulsion;
disodium EDTA, in about 0.1% by weight of the emulsion; and
sodium hydroxide;

a propellant; and

a purge gas.

In certain embodiments, the invention relates to a barrier repair formulation, consisting of:

an emulsion, wherein the emulsion consists of:
cetearyl alcohol, in about 2.4% by weight of the emulsion;
dicetyl phosphate, in about 2.4% by weight of the emulsion;
ceteareth-10 phosphate, in about 1.2% by weight of the emulsion;
steareth-10, in about 0.9% by weight of the emulsion;
Theobroma grandiflorum seed butter, in about 2% by weight of the emulsion;
white petrolatum, in about 1% by weight of the emulsion;
dimethicone, in about 1% by weight of the emulsion;
2-ethylhexyl palmitate, in about 6% by weight of the emulsion;
hydroxypropyl bispalmitamide MEA, in about 0.5% by weight of the emulsion;
tocopheryl acetate, in about 0.5% by weight of the emulsion;
water, in about 71.5% by weight of the emulsion;
propylene glycol, in about 2.5% by weight of the emulsion;
glycerol, in about 7.5% by weight of the emulsion;
sodium hyaluronate, in about 0.1% by weight of the emulsion;
methylparaben, in about 0.3% by weight of the emulsion;
propylparaben, in about 0.1% by weight of the emulsion;
disodium EDTA, in about 0.1% by weight of the emulsion; and
sodium hydroxide;

a propellant; and

a purge gas.

In certain embodiments, the invention relates to any one of the aforementioned barrier repair compositions (for example, a barrier repair emulsion or a barrier repair formulation) that does not comprise ceramide 2, ceramide 3, or a C₁₂-C₁₅ alkyl benzoate.

In certain embodiments, the invention relates to any one of the aforementioned barrier repair compositions, wherein the emulsifier or surfactant is selected from the group consisting of polysorbate 20, polysorbate 40, polysorbate 60, polysorbate 80, steareth-10, sodium dodecyl sulfate, lauryl dimethyl amine oxide, cetyltrimethylammonium bromide, polyethoxylated alcohols, polyoxyethylene sorbitan, octoxynol, N,N-dimethyldodecylamine-N-oxide, hexadecyltrimethylammonium bromide, polyoxyl 10 lauryl ether, sodium deoxycholate, sodium cholate, polyoxyl castor oil, nonylphenol ethoxylate, cyclodextrins, lecithin, dimethicone copolyol, lauramide DEA, cocamide DEA, cocamide MEA, oleyl betaine, cocamidopropyl betaine, cocamidopropyl phosphatidyl PG-dimonium chloride, dicetyl phosphate, ceteareth-10 phosphate, methylbenzethonium chloride, behentrimonium methosulfate-cetearyl alcohol, emulsifying wax, polyoxyethylene oleyl ether, PEG-40 stearate, cetostearyl alcohol, ceteareth-12, ceteareth-20, ceteareth-30, ceteareth alcohol, glyceryl stearate, PEG-100 stearate, glyceryl stearate, PEG-100 stearate, steareth-2, steareth-20, stearamidopropyl dimethylamine, and behentrimonium methosulfate, and combinations/mixtures thereof.

In certain embodiments, the invention relates to any one of the aforementioned barrier repair compositions, wherein the emulsifier or surfactant is selected from the group consisting of cetostearyl alcohol, dicetyl phosphate, ceteareth-10 phosphate, and steareth-10, and combinations/mixtures thereof.

In certain embodiments, the invention relates to any one of the aforementioned barrier repair compositions, wherein the emulsifier or surfactant is present in an amount from about 3% to about 11% by weight of the emulsion. In certain embodiments, the emulsifier or surfactant is present in an amount from about 5% to about 9% by weight of the emulsion. In certain embodiments, the emulsifier or surfactant is present in an amount of about 5%, about 5.5%, about 6%, about 6.5%, about 7%, about 7.5%, about 8%, about 8.5%, or about 9% by weight of the emulsion.

In certain embodiments, the invention relates to any one of the aforementioned barrier repair compositions, wherein the emulsifier or surfactant comprises cetostearyl alcohol (cetearyl alcohol). In certain embodiments, the cetostearyl alcohol is present in an amount from about 1% to about 4% by weight of the emulsion. In certain embodiments, the cetostearyl alcohol is present in an amount from about 2% to about 3% by weight of the emulsion. In certain embodiments, cetostearyl alcohol is present in about 2%, about 2.2%, about 2.4%, about 2.6%, about 2.8%, or about 3% by weight of the emulsion.

In certain embodiments, the invention relates to any one of the aforementioned barrier repair compositions, wherein the emulsifier or surfactant comprises dicetyl phosphate. In certain embodiments, the dicetyl phosphate is present in an amount from about 1% to about 4% by weight of the emulsion. In certain embodiments, the dicetyl phosphate is present in an amount from about 2% to about 3% by weight of the emulsion. In certain embodiments, dicetyl phosphate is present in about 2%, about 2.2%, about 2.4%, about 2.6%, about 2.8%, or about 3% by weight of the emulsion.

In certain embodiments, the invention relates to any one of the aforementioned barrier repair compositions, wherein the emulsifier or surfactant comprises ceteareth-10 phosphate. In certain embodiments, the ceteareth-10 phosphate is present in an amount from about 0.5% to about 2% by weight of the emulsion. In certain embodiments, the ceteareth-10 phosphate is present in an amount from about 1% to about 1.5% by weight of the emulsion. In certain embodiments, ceteareth-10 phosphate is present in about 1%, about 1.1%, about 1.2%, about 1.3%, about 1.4%, or about 1.5% by weight of the emulsion.

In certain embodiments, the invention relates to any one of the aforementioned barrier repair compositions, wherein the emulsifier or surfactant comprises steareth-10. In certain embodiments, the steareth-10 is present in an amount from about 0.4% to about 1.3% by weight of the emulsion. In certain embodiments, the steareth-10 is present in an amount from about 0.6% to about 1.1% by weight of the emulsion. In certain embodiments, steareth-10 is present in about 0.7%, about 0.8%, about 0.9%, about 1.0%, or about 1.1% by weight of the emulsion.

In certain embodiments, the invention relates to any one of the aforementioned barrier repair compositions, wherein the first moisturizer or first emollient is selected from the group consisting of white petrolatum, lactic acid, glycerol, propylene glycol, butylene glycol, sodium PCA, sodium hyaluronate, Carbowax 200, Carbowax 400, Carbowax 800, PPG-15 stearyl ether, lanolin alcohol, lanolin, lanolin derivatives, cholesterol, petrolatum, isostearyl neopentanoate, octyl stearate, mineral oil, isocetyl stearate, myristyl myristate, octyl dodecanol, 2-ethylhexyl palmitate (octyl palmitate), dimethicone, phenyl trimethicone, cyclomethicone, C₁₂-C₁₅ alkyl benzoates, dimethiconol, propylene glycol, Theobroma grandiflorum seed butter, ceramides, hydroxypropyl bispalmitamide MEA, hydroxypropyl bislauramide MEA, hydroxypropyl bisisostearamide MEA, 1,3-bis-(N-2-(hydroxyethyl)stearoylamino)-2-hydroxypropane, bis-hydroxyethyl tocopherylsuccinoylamido hydroxypropane, and dicaprylate/dicaprate, and combinations/mixtures thereof.

In certain embodiments, the invention relates to any one of the aforementioned barrier repair compositions, wherein the first moisturizer or first emollient is selected from the group consisting of white petrolatum, dimethicone, 2-ethylhexyl palmitate, hydroxypropyl bispalmitate MEA, Theobroma grandiflorum seed butter, and combinations/mixtures thereof.

In certain embodiments, the invention relates to any one of the aforementioned barrier repair compositions, wherein the first moisturizer or first emollient is present in an amount from about 5% to about 15% by weight of the emulsion. In certain embodiments, the first moisturizer or first emollient is present in an amount from about 7% to about 13% by weight of the emulsion. In certain embodiments, the first moisturizer or first emollient is present in an amount of about 7%, about 7.5%, about 8%, about 8.5%, about 9%, about 9.5%, about 10%, about 10.5%, about 11%, about 11.5%, about 12%, about 12.5%, or about 13% by weight of the emulsion.

In certain embodiments, the invention relates to any one of the aforementioned barrier repair compositions, wherein the first moisturizer or first emollient comprises Theobroma grandiflorum seed butter. In certain embodiments, the Theobroma grandiflorum seed butter is present in an amount from about 1% to about 3% by weight of the emulsion. In certain embodiments, the Theobroma grandiflorum seed butter is present in an amount from about 1.5% to about 2.5% by weight of the emulsion. In certain embodiments, Theobroma grandiflorum seed butter is present in about 1.5%, about 1.6%, about 1.7%, about 1.8%, about 1.9%, about 2.0%, about 2.1%, about 2.2%, about 2.3%, about 2.4%, or about 2.5% by weight of the emulsion.

In certain embodiments, the invention relates to any one of the aforementioned barrier repair compositions, wherein the first moisturizer or first emollient comprises white petrolatum. In certain embodiments, the white petrolatum is present in an amount from about 0.5% to about 1.5% by weight of the emulsion. In certain embodiments, the white petrolatum is present in an amount from about 0.7% to about 1.3% by weight of the emulsion. In certain embodiments, white petrolatum is present in about 0.7%, about 0.8%, about 0.9%, about 1.0%, about 1.1%, about 1.2%, or about 1.3% by weight of the emulsion.

In certain embodiments, the invention relates to any one of the aforementioned barrier repair compositions, wherein the first moisturizer or first emollient comprises dimethicone. In certain embodiments, the dimethicone is present in an amount from about 0.5% to about 1.5% by weight of the emulsion. In certain embodiments, the dimethicone is present in an amount from about 0.7% to about 1.3% by weight of the emulsion. In certain embodiments, dimethicone is present in about 0.7%, about 0.8%, about 0.9%, about 1.0%, about 1.1%, about 1.2%, or about 1.3% by weight of the emulsion.

In certain embodiments, the invention relates to any one of the aforementioned barrier repair compositions, wherein the first moisturizer or first emollient comprises 2-ethylhexyl palmitate. In certain embodiments, the 2-ethylhexyl palmitate is present in an amount from about 3% to about 9% by weight of the emulsion. In certain embodiments, the 2-ethylhexyl palmitate is present in an amount from about 4% to about 8% by weight of the emulsion. In certain embodiments, the 2-ethylhexyl palmitate is present in about 4%, about 4.5%, about 5%, about 5.5%, about 6%, about 6.5%, about 7%, about 7.5%, or about 8% by weight of the emulsion.

In certain embodiments, the invention relates to any one of the aforementioned barrier repair compositions, wherein the first moisturizer or first emollient comprises hydroxypropyl bispalmitamide MEA. In certain embodiments, the hydroxypropyl bispalmitamide MEA is present in an amount from about 0.2% to about 0.8% by weight of the emulsion. In certain embodiments, the hydroxypropyl bispalmitamide MEA is present in an amount from about 0.3% to about 0.7% by weight of the emulsion. In certain embodiments, the hydroxypropyl bispalmitamide MEA is present in about 0.35%, about 0.4%, about 0.45%, about 0.5%, about 0.55%, about 0.6%, or about 0.65% by weight of the emulsion.

In certain embodiments, the invention relates to any one of the aforementioned barrier repair compositions, wherein the first antioxidant or first preservative is selected from the group consisting of imidazolidinyl urea, diazolidinyl urea, phenoxyethanol, sodium methyl paraben, methylparaben, ethylparaben, propylparaben, potassium sorbate, sodium benzoate, sorbic acid, benzoic acid, formaldehyde, citric acid, sodium citrate, chlorine dioxide, benzalkonium chloride, benzethonium chloride, cetrimide, dequalinium chloride, cetylpyridinium chloride, phenylmercuric nitrate, phenylmercuric acetate, thimerosal, chlorobutanol, dichlorobenzyl alcohol, phenylethyl alcohol, benzyl alcohol, ascorbic acid and its esters, sodium bisulfite, butylated hydroxytoluene, butylated hydroxyanisole, tocopherols (such as α-tocopherol), tocopheryl acetate, sodium ascorbate/ascorbic acid, ascorbyl palmitate, propyl gallate, disodium EDTA, and sodium citrate, and combinations/mixtures thereof.

In certain embodiments, the invention relates to any one of the aforementioned barrier repair compositions, wherein the first antioxidant or first preservative comprises tocopheryl acetate.

In certain embodiments, the invention relates to any one of the aforementioned barrier repair compositions, wherein the first antioxidant or first preservative is present in an amount from about 0.2% to about 0.8% by weight of the emulsion. In certain embodiments, the first antioxidant or first preservative is present in an amount from about 0.4% to about 0.6% by weight of the emulsion. In certain embodiments, the first antioxidant or first preservative is present in an amount of about 0.4%, about 0.45%, about 0.5%, about 0.55%, or about 0.6% by weight of the emulsion.

In certain embodiments, the invention relates to any one of the aforementioned barrier repair compositions, wherein the first antioxidant or first preservative comprises tocopheryl acetate. In certain embodiments, the tocopheryl acetate is present in an amount from about 0.2% to about 0.8% by weight of the emulsion. In certain embodiments, the tocopheryl acetate is present in an amount from about 0.4% to about 0.6% by weight of the emulsion. In certain embodiments, tocopheryl acetate is present in about 0.4%, about 0.45%, about 0.5%, about 0.55%, or about 0.6% by weight of the emulsion.

In certain embodiments, the invention relates to a barrier repair emulsion, comprising:
an oil phase, wherein the oil phase comprises an emulsifier or surfactant, a first moisturizer or first emollient, and a first antioxidant or first preservative; and
an aqueous phase, wherein the aqueous phase comprises a vehicle, a second moisturizer or second emollient, a second antioxidant or second preservative, and a pH adjuster.

In certain embodiments, the invention relates to a barrier repair emulsion, consisting essentially of:
an oil phase, wherein the oil phase consists essentially of an emulsifier or surfactant, a first moisturizer or first emollient, and a first antioxidant or first preservative; and
an aqueous phase, wherein the aqueous phase consists essentially of a vehicle, a second moisturizer or second emollient, a second antioxidant or second preservative, and a pH adjuster.

In certain embodiments, the invention relates to a barrier repair emulsion, consisting of:
an oil phase, wherein the oil phase consists of an emulsifier or surfactant, a first moisturizer or first emollient, and a first antioxidant or first preservative; and
an aqueous phase, wherein the aqueous phase consists of a vehicle, a second moisturizer or second emollient, a second antioxidant or second preservative, and a pH adjuster.

In certain embodiments, the invention relates to a barrier repair emulsion, comprising:
cetearyl alcohol, from about 1% to about 4% by weight of the emulsion;
dicetyl phosphate, from about 1% to about 4% by weight of the emulsion;
ceteareth-10 phosphate, from about 0.5% to about 2% by weight of the emulsion;
steareth-10, from about 0.4% to about 1.3% by weight of the emulsion;
Theobroma grandiflorum seed butter, from about 1% to about 3% by weight of the emulsion;
white petrolatum, from about 0.5% to about 1.5% by weight of the emulsion;
dimethicone, from about 0.5% to about 1.5% by weight of the emulsion;
2-ethylhexyl palmitate, from about 3% to about 9% by weight of the emulsion;
tocopheryl acetate, from about 0.2% to about 0.8% by weight of the emulsion;
water, from about 55% to about 85% by weight of the emulsion;
propylene glycol, from about 1.2% to about 3.8% by weight of the emulsion;
glycerol, from about 4% to about 11% by weight of the emulsion;
sodium hyaluronate, from about 0.05% to about 0.2% by weight of the emulsion;
methylparaben, from about 0.1% to about 0.5% by weight of the emulsion;
propylparaben, from about 0.05% to about 0.2% by weight of the emulsion;
disodium EDTA, from about 0.05% to about 0.2% by weight of the emulsion; and
sodium hydroxide.

In certain embodiments, the invention relates to a barrier repair emulsion, consisting essentially of:
cetearyl alcohol, from about 1% to about 4% by weight of the emulsion;
dicetyl phosphate, from about 1% to about 4% by weight of the emulsion;
ceteareth-10 phosphate, from about 0.5% to about 2% by weight of the emulsion;
steareth-10, from about 0.4% to about 1.3% by weight of the emulsion;
Theobroma grandiflorum seed butter, from about 1% to about 3% by weight of the emulsion;
white petrolatum, from about 0.5% to about 1.5% by weight of the emulsion;
dimethicone, from about 0.5% to about 1.5% by weight of the emulsion;
2-ethylhexyl palmitate, from about 3% to about 9% by weight of the emulsion;
tocopheryl acetate, from about 0.2% to about 0.8% by weight of the emulsion;
water, from about 55% to about 85% by weight of the emulsion;
propylene glycol, from about 1.2% to about 3.8% by weight of the emulsion;
glycerol, from about 4% to about 11% by weight of the emulsion;
sodium hyaluronate, from about 0.05% to about 0.2% by weight of the emulsion;
methylparaben, from about 0.1% to about 0.5% by weight of the emulsion;
propylparaben, from about 0.05% to about 0.2% by weight of the emulsion;
disodium EDTA, from about 0.05% to about 0.2% by weight of the emulsion; and
sodium hydroxide.

In certain embodiments, the invention relates to an emulsion, consisting of:
cetearyl alcohol, from about 1% to about 4% by weight of the emulsion;
dicetyl phosphate, from about 1% to about 4% by weight of the emulsion;
ceteareth-10 phosphate, from about 0.5% to about 2% by weight of the emulsion;
steareth-10, from about 0.4% to about 1.3% by weight of the emulsion;
Theobroma grandiflorum seed butter, from about 1% to about 3% by weight of the emulsion;
white petrolatum, from about 0.5% to about 1.5% by weight of the emulsion;
dimethicone, from about 0.5% to about 1.5% by weight of the emulsion;
2-ethylhexyl palmitate, from about 3% to about 9% by weight of the emulsion;
tocopheryl acetate, from about 0.2% to about 0.8% by weight of the emulsion;
water, from about 55% to about 85% by weight of the emulsion;
propylene glycol, from about 1.2% to about 3.8% by weight of the emulsion;
glycerol, from about 4% to about 11% by weight of the emulsion;
sodium hyaluronate, from about 0.05% to about 0.2% by weight of the emulsion;
methylparaben, from about 0.1% to about 0.5% by weight of the emulsion;
propylparaben, from about 0.05% to about 0.2% by weight of the emulsion;
disodium EDTA, from about 0.05% to about 0.2% by weight of the emulsion; and
sodium hydroxide.

In certain embodiments, the invention relates to a barrier repair emulsion, comprising:
cetearyl alcohol, in about 2.4% by weight of the emulsion;
dicetyl phosphate, in about 2.4% by weight of the emulsion;
ceteareth-10 phosphate, in about 1.2% by weight of the emulsion;
steareth-10, in about 0.9% by weight of the emulsion;
Theobroma grandiflorum seed butter, in about 2% by weight of the emulsion;
white petrolatum, in about 1% by weight of the emulsion;
dimethicone, in about 1% by weight of the emulsion;
2-ethylhexyl palmitate, in about 6% by weight of the emulsion;
tocopheryl acetate, in about 0.5% by weight of the emulsion;
water, in about 72.0% by weight of the emulsion;
propylene glycol, in about 2.5% by weight of the emulsion;
glycerol, in about 7.5% by weight of the emulsion;
sodium hyaluronate, in about 0.1% by weight of the emulsion;
methylparaben, in about 0.3% by weight of the emulsion;
propylparaben, in about 0.1% by weight of the emulsion;
disodium EDTA, in about 0.1% by weight of the emulsion; and
sodium hydroxide.

In certain embodiments, the invention relates to a barrier repair emulsion, consisting essentially of:
cetearyl alcohol, in about 2.4% by weight of the emulsion;
dicetyl phosphate, in about 2.4% by weight of the emulsion;
ceteareth-10 phosphate, in about 1.2% by weight of the emulsion;
steareth-10, in about 0.9% by weight of the emulsion;
Theobroma grandiflorum seed butter, in about 2% by weight of the emulsion;
white petrolatum, in about 1% by weight of the emulsion;
dimethicone, in about 1% by weight of the emulsion;
2-ethylhexyl palmitate, in about 6% by weight of the emulsion;
tocopheryl acetate, in about 0.5% by weight of the emulsion;
water, in about 72.0% by weight of the emulsion;
propylene glycol, in about 2.5% by weight of the emulsion;
glycerol, in about 7.5% by weight of the emulsion;
sodium hyaluronate, in about 0.1% by weight of the emulsion;
methylparaben, in about 0.3% by weight of the emulsion;
propylparaben, in about 0.1% by weight of the emulsion;
disodium EDTA, in about 0.1% by weight of the emulsion; and
sodium hydroxide.

In certain embodiments, the invention relates to an emulsion, consisting of:
cetearyl alcohol, in about 2.4% by weight of the emulsion;
dicetyl phosphate, in about 2.4% by weight of the emulsion;
ceteareth-10 phosphate, in about 1.2% by weight of the emulsion;
steareth-10, in about 0.9% by weight of the emulsion;
Theobroma grandiflorum seed butter, in about 2% by weight of the emulsion;
white petrolatum, in about 1% by weight of the emulsion;
dimethicone, in about 1% by weight of the emulsion;
2-ethylhexyl palmitate, in about 6% by weight of the emulsion;
tocopheryl acetate, in about 0.5% by weight of the emulsion;
water, in about 72.0% by weight of the emulsion;
propylene glycol, in about 2.5% by weight of the emulsion;
glycerol, in about 7.5% by weight of the emulsion;
sodium hyaluronate, in about 0.1% by weight of the emulsion;
methylparaben, in about 0.3% by weight of the emulsion;
propylparaben, in about 0.1% by weight of the emulsion;
disodium EDTA, in about 0.1% by weight of the emulsion; and
sodium hydroxide.

In certain embodiments, the invention relates to a barrier repair formulation, comprising:

an emulsion, wherein the emulsion comprises:
an oil phase, wherein the oil phase comprises an emulsifier or surfactant, a first moisturizer or first emollient, and a first antioxidant or first preservative; and
an aqueous phase, wherein the aqueous phase comprises a vehicle, a second moisturizer or second emollient, a second antioxidant or second preservative, and a pH adjuster;

a propellant; and

a purge gas.

In certain embodiments, the invention relates to a barrier repair formulation, consisting essentially of:

an emulsion, wherein the emulsion consists essentially of:
an oil phase, wherein the oil phase consists essentially of an emulsifier or surfactant, a first moisturizer or first emollient, and a first antioxidant or first preservative; and
an aqueous phase, wherein the aqueous phase consists essentially of a vehicle, a second moisturizer or second emollient, a second antioxidant or second preservative, and a pH adjuster;

a propellant; and

a purge gas.

In certain embodiments, the invention relates to a barrier repair formulation, consisting of:

an emulsion, wherein the emulsion consists of:
an oil phase, wherein the oil phase consists of an emulsifier or surfactant, a first moisturizer or first emollient, and a first antioxidant or first preservative; and
an aqueous phase, wherein the aqueous phase consists of a vehicle, a second moisturizer or second emollient, a second antioxidant or second preservative, and a pH adjuster;

a propellant; and

a purge gas.

In certain embodiments, the invention relates to a barrier repair formulation, comprising:

an emulsion, wherein the emulsion comprises:
cetearyl alcohol, from about 1% to about 4% by weight of the emulsion; dicetyl phosphate, from about 1% to about 4% by weight of the emulsion;
ceteareth-10 phosphate, from about 0.5% to about 2% by weight of the emulsion;
steareth-10, from about 0.4% to about 1.3% by weight of the emulsion;
Theobroma grandiflorum seed butter, from about 1% to about 3% by weight of the emulsion;
white petrolatum, from about 0.5% to about 1.5% by weight of the emulsion;
dimethicone, from about 0.5% to about 1.5% by weight of the emulsion;
2-ethylhexyl palmitate, from about 3% to about 9% by weight of the emulsion;
tocopheryl acetate, from about 0.2% to about 0.8% by weight of the emulsion;
water, from about 55% to about 85% by weight of the emulsion;
propylene glycol, from about 1.2% to about 3.8% by weight of the emulsion;
glycerol, from about 4% to about 11% by weight of the emulsion;
sodium hyaluronate, from about 0.05% to about 0.2% by weight of the emulsion;
methylparaben, from about 0.1% to about 0.5% by weight of the emulsion;
propylparaben, from about 0.05% to about 0.2% by weight of the emulsion;
disodium EDTA, from about 0.05% to about 0.2% by weight of the emulsion; and
sodium hydroxide;

a propellant; and

a purge gas.

In certain embodiments, the invention relates to a barrier repair formulation, consisting essentially of:

an emulsion, wherein the emulsion consists essentially of:
cetearyl alcohol, from about 1% to about 4% by weight of the emulsion;
dicetyl phosphate, from about 1% to about 4% by weight of the emulsion;
ceteareth-10 phosphate, from about 0.5% to about 2% by weight of the emulsion;
steareth-10, from about 0.4% to about 1.3% by weight of the emulsion;
Theobroma grandiflorum seed butter, from about 1% to about 3% by weight of the emulsion;
white petrolatum, from about 0.5% to about 1.5% by weight of the emulsion;
dimethicone, from about 0.5% to about 1.5% by weight of the emulsion;
2-ethylhexyl palmitate, from about 3% to about 9% by weight of the emulsion;
tocopheryl acetate, from about 0.2% to about 0.8% by weight of the emulsion;
water, from about 55% to about 85% by weight of the emulsion;
propylene glycol, from about 1.2% to about 3.8% by weight of the emulsion;
glycerol, from about 4% to about 11% by weight of the emulsion;
sodium hyaluronate, from about 0.05% to about 0.2% by weight of the emulsion;
methylparaben, from about 0.1% to about 0.5% by weight of the emulsion;
propylparaben, from about 0.05% to about 0.2% by weight of the emulsion;
disodium EDTA, from about 0.05% to about 0.2% by weight of the emulsion; and
sodium hydroxide;

a propellant; and

a purge gas.

In certain embodiments, the invention relates to a barrier repair formulation, consisting of:

an emulsion, wherein the emulsion consists of:
cetearyl alcohol, from about 1% to about 4% by weight of the emulsion;
dicetyl phosphate, from about 1% to about 4% by weight of the emulsion;
ceteareth-10 phosphate, from about 0.5% to about 2% by weight of the emulsion;
steareth-10, from about 0.4% to about 1.3% by weight of the emulsion;
Theobroma grandiflorum seed butter, from about 1% to about 3% by weight of the emulsion;
white petrolatum, from about 0.5% to about 1.5% by weight of the emulsion;
dimethicone, from about 0.5% to about 1.5% by weight of the emulsion;
2-ethylhexyl palmitate, from about 3% to about 9% by weight of the emulsion;
tocopheryl acetate, from about 0.2% to about 0.8% by weight of the emulsion;
water, from about 55% to about 85% by weight of the emulsion;
propylene glycol, from about 1.2% to about 3.8% by weight of the emulsion;
glycerol, from about 4% to about 11% by weight of the emulsion;
sodium hyaluronate, from about 0.05% to about 0.2% by weight of the emulsion;
methylparaben, from about 0.1% to about 0.5% by weight of the emulsion;
propylparaben, from about 0.05% to about 0.2% by weight of the emulsion;
disodium EDTA, from about 0.05% to about 0.2% by weight of the emulsion; and
sodium hydroxide;

a propellant; and

a purge gas.

In certain embodiments, the invention relates to a barrier repair formulation, comprising:

an emulsion, wherein the emulsion comprises:
cetearyl alcohol, in about 2.4% by weight of the emulsion;
dicetyl phosphate, in about 2.4% by weight of the emulsion;
ceteareth-10 phosphate, in about 1.2% by weight of the emulsion;
steareth-10, in about 0.9% by weight of the emulsion;
Theobroma grandiflorum seed butter, in about 2% by weight of the emulsion;
white petrolatum, in about 1% by weight of the emulsion;
dimethicone, in about 1% by weight of the emulsion;
2-ethylhexyl palmitate, in about 6% by weight of the emulsion;
tocopheryl acetate, in about 0.5% by weight of the emulsion;
water, in about 72.0% by weight of the emulsion;
propylene glycol, in about 2.5% by weight of the emulsion;
glycerol, in about 7.5% by weight of the emulsion;
sodium hyaluronate, in about 0.1% by weight of the emulsion;
methylparaben, in about 0.3% by weight of the emulsion;
propylparaben, in about 0.1% by weight of the emulsion;
disodium EDTA, in about 0.1% by weight of the emulsion; and
sodium hydroxide;

a propellant; and

a purge gas.

In certain embodiments, the invention relates to a barrier repair formulation, consisting essentially of:

an emulsion, wherein the emulsion consists essentially of:
cetearyl alcohol, in about 2.4% by weight of the emulsion;
dicetyl phosphate, in about 2.4% by weight of the emulsion;
ceteareth-10 phosphate, in about 1.2% by weight of the emulsion;
steareth-10, in about 0.9% by weight of the emulsion;
Theobroma grandiflorum seed butter, in about 2% by weight of the emulsion;
white petrolatum, in about 1% by weight of the emulsion;
dimethicone, in about 1% by weight of the emulsion;
2-ethylhexyl palmitate, in about 6% by weight of the emulsion;
tocopheryl acetate, in about 0.5% by weight of the emulsion;
water, in about 72.0% by weight of the emulsion;
propylene glycol, in about 2.5% by weight of the emulsion;
glycerol, in about 7.5% by weight of the emulsion;
sodium hyaluronate, in about 0.1% by weight of the emulsion;
methylparaben, in about 0.3% by weight of the emulsion;
propylparaben, in about 0.1% by weight of the emulsion;
disodium EDTA, in about 0.1% by weight of the emulsion; and
sodium hydroxide;

a propellant; and

a purge gas.

In certain embodiments, the invention relates to a barrier repair formulation, consisting of:

an emulsion, wherein the emulsion consists of:
cetearyl alcohol, in about 2.4% by weight of the emulsion;
dicetyl phosphate, in about 2.4% by weight of the emulsion;
ceteareth-10 phosphate, in about 1.2% by weight of the emulsion;
steareth-10, in about 0.9% by weight of the emulsion;
Theobroma grandiflorum seed butter, in about 2% by weight of the emulsion;
white petrolatum, in about 1% by weight of the emulsion;
dimethicone, in about 1% by weight of the emulsion;
2-ethylhexyl palmitate, in about 6% by weight of the emulsion;
tocopheryl acetate, in about 0.5% by weight of the emulsion;
water, in about 72.0% by weight of the emulsion;
propylene glycol, in about 2.5% by weight of the emulsion;
glycerol, in about 7.5% by weight of the emulsion;
sodium hyaluronate, in about 0.1% by weight of the emulsion;
methylparaben, in about 0.3% by weight of the emulsion;
propylparaben, in about 0.1% by weight of the emulsion;
disodium EDTA, in about 0.1% by weight of the emulsion; and
sodium hydroxide;

a propellant; and

a purge gas.

In certain embodiments, the invention relates to any one of the aforementioned barrier repair compositions (for example, a barrier repair emulsion or a barrier repair formulation) that does not comprise a C₁₂-C₁₅ alkyl benzoate, ceramide 2, ceramide 3, or hydroxypropyl bispalmitamide MEA.

In certain embodiments, the invention relates to any one of the aforementioned barrier repair compositions, wherein the emulsifier or surfactant is selected from the group consisting of polysorbate 20, polysorbate 40, polysorbate 60, polysorbate 80, steareth-10, sodium dodecyl sulfate, lauryl dimethyl amine oxide, cetyltrimethylammonium bromide, polyethoxylated alcohols, polyoxyethylene sorbitan, octoxynol, N,N-dimethyldodecylamine-N-oxide, hexadecyltrimethylammonium bromide, polyoxyl 10 lauryl ether, sodium deoxycholate, sodium cholate, polyoxyl castor oil, nonylphenol ethoxylate, cyclodextrins, lecithin, dimethicone copolyol, lauramide DEA, cocamide DEA, cocamide MEA, oleyl betaine, cocamidopropyl betaine, cocamidopropyl phosphatidyl PG-dimonium chloride, dicetyl phosphate, ceteareth-10 phosphate, methylbenzethonium chloride, behentrimonium methosulfate-cetearyl alcohol, emulsifying wax, polyoxyethylene oleyl ether, PEG-40 stearate, cetostearyl alcohol, ceteareth-12, ceteareth-20, ceteareth-30, ceteareth alcohol, glyceryl stearate, PEG-100 stearate, glyceryl stearate, PEG-100 stearate, steareth-2, steareth-20, stearamidopropyl dimethylamine, and behentrimonium methosulfate, and combinations/mixtures thereof.

In certain embodiments, the invention relates to any one of the aforementioned barrier repair compositions, wherein the emulsifier or surfactant is selected from the group consisting of cetostearyl alcohol, dicetyl phosphate, ceteareth-10 phosphate, and steareth-10, and combinations/mixtures thereof.

In certain embodiments, the invention relates to any one of the aforementioned barrier repair compositions, wherein the emulsifier or surfactant is present in an amount from about 3% to about 11% by weight of the emulsion. In certain embodiments, the emulsifier or surfactant is present in an amount from about 5% to about 9% by weight of the emulsion. In certain embodiments, the emulsifier or surfactant is present in an amount of about 5%, about 5.5%, about 6%, about 6.5%, about 7%, about 7.5%, about 8%, about 8.5%, or about 9% by weight of the emulsion.

In certain embodiments, the invention relates to any one of the aforementioned barrier repair compositions, wherein the emulsifier or surfactant comprises cetostearyl alcohol (cetearyl alcohol). In certain embodiments, the cetostearyl alcohol is present in an amount from about 1% to about 4% by weight of the emulsion. In certain embodiments, the cetostearyl alcohol is present in an amount from about 2% to about 3% by weight of the emulsion. In certain embodiments, cetostearyl alcohol is present in about 2%, about 2.2%, about 2.4%, about 2.6%, about 2.8%, or about 3% by weight of the emulsion.

In certain embodiments, the invention relates to any one of the aforementioned barrier repair compositions, wherein the emulsifier or surfactant comprises dicetyl phosphate. In certain embodiments, the dicetyl phosphate is present in an amount from about 1% to about 4% by weight of the emulsion. In certain embodiments, the dicetyl phosphate is present in an amount from about 2% to about 3% by weight of the emulsion. In certain embodiments, dicetyl phosphate is present in about 2%, about 2.2%, about 2.4%, about 2.6%, about 2.8%, or about 3% by weight of the emulsion.

In certain embodiments, the invention relates to any one of the aforementioned barrier repair compositions, wherein the emulsifier or surfactant comprises ceteareth-10 phosphate. In certain embodiments, the ceteareth-10 phosphate is present in an amount from about 0.5% to about 2% by weight of the emulsion. In certain embodiments, the ceteareth-10 phosphate is present in an amount from about 1% to about 1.5% by weight of the emulsion. In certain embodiments, ceteareth-10 phosphate is present in about 1%, about 1.1%, about 1.2%, about 1.3%, about 1.4%, or about 1.5% by weight of the emulsion.

In certain embodiments, the invention relates to any one of the aforementioned barrier repair compositions, wherein the emulsifier or surfactant comprises steareth-10. In certain embodiments, the steareth-10 is present in an amount from about 0.4% to about 1.3% by weight of the emulsion. In certain embodiments, the steareth-10 is present in an amount from about 0.6% to about 1.1% by weight of the emulsion. In certain embodiments, steareth-10 is present in about 0.7%, about 0.8%, about 0.9%, about 1.0%, or about 1.1% by weight of the emulsion.

In certain embodiments, the invention relates to any one of the aforementioned barrier repair compositions, wherein the first moisturizer or first emollient is selected from the group consisting of white petrolatum, lactic acid, glycerol, propylene glycol, butylene glycol, sodium PCA, sodium hyaluronate, Carbowax 200, Carbowax 400, Carbowax 800, PPG-15 stearyl ether, lanolin alcohol, lanolin, lanolin derivatives, cholesterol, petrolatum, isostearyl neopentanoate, octyl stearate, mineral oil, isocetyl stearate, myristyl myristate, octyl dodecanol, 2-ethylhexyl palmitate (octyl palmitate), dimethicone, phenyl trimethicone, cyclomethicone, C₁₂-C₁₅ alkyl benzoates, dimethiconol, propylene glycol, Theobroma Graniflorum seed butter, ceramides, hydroxypropyl bispalmitamide MEA, hydroxypropyl bislauramide MEA, hydroxypropyl bisisostearamide MEA, 1,3-bis-(N-2-(hydroxyethyl)stearoylamino)-2-hydroxypropane, bis-hydroxyethyl tocopherylsuccinoylamido hydroxypropane, and dicaprylate/dicaprate, and combinations/mixtures thereof.

In certain embodiments, the invention relates to any one of the aforementioned barrier repair compositions, wherein the first moisturizer or first emollient is selected from the group consisting of white petrolatum, dimethicone, 2-ethylhexyl palmitate, Theobroma grandiflorum seed butter, and combinations/mixtures thereof.

In certain embodiments, the invention relates to any one of the aforementioned barrier repair compositions, wherein the first moisturizer or first emollient is present in an amount from about 5% to about 15% by weight of the emulsion. In certain embodiments, the first moisturizer or first emollient is present in an amount from about 7% to about 13% by weight of the emulsion. In certain embodiments, the first moisturizer or first emollient is present in an amount of about 7%, about 7.5%, about 8%, about 8.5%, about 9%, about 9.5%, about 10%, about 10.5%, about 11%, about 11.5%, about 12%, about 12.5%, or about 13% by weight of the emulsion.

In certain embodiments, the invention relates to any one of the aforementioned barrier repair compositions, wherein the first moisturizer or first emollient comprises Theobroma grandiflorum seed butter. In certain embodiments, the Theobroma grandiflorum seed butter is present in an amount from about 1% to about 3% by weight of the emulsion. In certain embodiments, the Theobroma grandiflorum seed butter is present in an amount from about 1.5% to about 2.5% by weight of the emulsion. In certain embodiments, Theobroma grandiflorum seed butter is present in about 1.5%, about 1.6%, about 1.7%, about 1.8%, about 1.9%, about 2.0%, about 2.1%, about 2.2%, about 2.3%, about 2.4%, or about 2.5% by weight of the emulsion.

In certain embodiments, the invention relates to any one of the aforementioned barrier repair compositions, wherein the first moisturizer or first emollient comprises white petrolatum. In certain embodiments, the white petrolatum is present in an amount from about 0.5% to about 1.5% by weight of the emulsion. In certain embodiments, the white petrolatum is present in an amount from about 0.7% to about 1.3% by weight of the emulsion. In certain embodiments, white petrolatum is present in about 0.7%, about 0.8%, about 0.9%, about 1.0%, about 1.1%, about 1.2%, or about 1.3% by weight of the emulsion.

In certain embodiments, the invention relates to any one of the aforementioned barrier repair compositions, wherein the first moisturizer or first emollient comprises dimethicone. In certain embodiments, the dimethicone is present in an amount from about 0.5% to about 1.5% by weight of the emulsion. In certain embodiments, the dimethicone is present in an amount from about 0.7% to about 1.3% by weight of the emulsion. In certain embodiments, dimethicone is present in about 0.7%, about 0.8%, about 0.9%, about 1.0%, about 1.1%, about 1.2%, or about 1.3% by weight of the emulsion.

In certain embodiments, the invention relates to any one of the aforementioned barrier repair compositions, wherein the first moisturizer or first emollient comprises 2-ethylhexyl palmitate. In certain embodiments, the 2-ethylhexyl palmitate is present in an amount from about 3% to about 9% by weight of the emulsion. In certain embodiments, the 2-ethylhexyl palmitate is present in an amount from about 4% to about 8% by weight of the emulsion. In certain embodiments, the 2-ethylhexyl palmitate is present in about 4%, about 4.5%, about 5%, about 5.5%, about 6%, about 6.5%, about 7%, about 7.5%, or about 8% by weight of the emulsion.

In certain embodiments, the invention relates to any one of the aforementioned barrier repair compositions, wherein the first antioxidant or first preservative is selected from the group consisting of imidazolidinyl urea, diazolidinyl urea, phenoxyethanol, sodium methyl paraben, methylparaben, ethylparaben, propylparaben, potassium sorbate, sodium benzoate, sorbic acid, benzoic acid, formaldehyde, citric acid, sodium citrate, chlorine dioxide, benzalkonium chloride, benzethonium chloride, cetrimide, dequalinium chloride, cetylpyridinium chloride, phenylmercuric nitrate, phenylmercuric acetate, thimerosal, chlorobutanol, dichlorobenzyl alcohol, phenylethyl alcohol, benzyl alcohol, ascorbic acid and its esters, sodium bisulfite, butylated hydroxytoluene, butylated hydroxyanisole, tocopherols (such as α-tocopherol), tocopheryl acetate, sodium ascorbate/ascorbic acid, ascorbyl palmitate, propyl gallate, disodium EDTA, and sodium citrate, and combinations/mixtures thereof.

In certain embodiments, the invention relates to any one of the aforementioned barrier repair compositions, wherein the first antioxidant or first preservative comprises tocopheryl acetate.

In certain embodiments, the invention relates to any one of the aforementioned barrier repair compositions, wherein the first antioxidant or first preservative is present in an amount from about 0.2% to about 0.8% by weight of the emulsion. In certain embodiments, the first antioxidant or first preservative is present in an amount from about 0.4% to about 0.6% by weight of the emulsion. In certain embodiments, the first antioxidant or first preservative is present in an amount of about 0.4%, about 0.45%, about 0.5%, about 0.55%, or about 0.6% by weight of the emulsion.

In certain embodiments, the invention relates to any one of the aforementioned barrier repair compositions, wherein the first antioxidant or first preservative comprises tocopheryl acetate. In certain embodiments, the tocopheryl acetate is present in an amount from about 0.2% to about 0.8% by weight of the emulsion. In certain embodiments, the tocopheryl acetate is present in an amount from about 0.4% to about 0.6% by weight of the emulsion. In certain embodiments, tocopheryl acetate is present in about 0.4%, about 0.45%, about 0.5%, about 0.55%, or about 0.6% by weight of the emulsion.

In one embodiment, the barrier repair compositions are commercially available creams, lotions, sprays, gels, foams or ointments. Examples of commercially available barrier repair compositions include:
1. Atopiclair produced by Graceway Pharmaceuticals (includes Glycyrrhetinic acid, Hyaluronic acid, shea butter, triglycerides, and polyunsaturated fatty acids)
2. Biafine produced by Ortho Dermatologics (purified water, liquid paraffin, ethylene glycol monostearate, stearic acid, propylene glycol, paraffin wax, squalane, avocado oil, trolamine/sodium alginate, triethanolamine, cetyl palmitate, methylparaben (sodium salt), sorbic acid (potassium salt), propylparaben (sodium salt), and fragrance)
3. Eletone produced by Ferndale Laboratories (petrolatum, purified water, mineral oil, cetostearyl alcohol, ceteth-20, citric acid, sodium citrate, propylparaben, and butylparaben.)
4. Epiceram produced by Promius Pharma (Capric Acid, Cholesterol, Citric Acid, Conjugated Linoleic Acid, Dimethicone, Disodium EDTA, E. Cerifera (Candelilla) Wax, Food Starch Modified Corn Syrup Solids, Glycerin, Glyceryl Stearate, Hydroxypropyl Bispalmitamide MEA (Ceramide), Palmitic Acid, PEG-100 Stearate, Petrolatum, Phenoxyethanol, Potassium Hydroxide, Purified Water, Sorbic Acid, Squalane, Xanthan Gum)
5. Hylira produced by Hawthorn Pharmaceuticals (includes water, sodium hyaluronate)
6. Mimyx produced by Stiefel Laboratories (purified water, olive oil, glycerin, pentylene glycol, palm glycerides, vegetable oil, hydrogenated lecithin, squalane, betaine, palmitamide MEA, sarcosine, acetamide MEA, hydroxyethyl cellulose, sodium carbomer, carbomer, xanthan gum)
7. Tetrix produced by Valeant Pharmaceuticals (aluminum magnesium hydroxide stearate, cetyl dimethicone copolyol, cyclomethicone, dimethicone, hexyl laurate, polyglyceryl-4-isostearate, purified water, and sodium chloride, phenoxyethanol and propylparaben)
8. Neosalus produced by Quinnova Pharmaceuticals (Carbomer 940 (benzene-free crosslinked polyacrylic acid), dimethicone, ethyl paraben, glycerine, methyl paraben, phenoxyethanol, polysorbate 20, polyvinylpyrrolidone, propylene glycol, propyl paraben, purified water, stearic acid, triethanolamine, butane and propane (propellants); fragrance-free)
9. Hylatopic produced by Onset Therapeutics (Water, Hydrofluorocarbon 134a (propellant), Glycerin, Ethylhexyl Palmitate, Cetearyl Alcohol, Propylene Glycol, Dicetyl Phosphate, Theobroma Grandiflorum Seed Butter, Petrolatum, Dimethicone, Steareth-10, Ceteareth-10 Phosphate, Tocopheryl Acetate, Methylparaben, Disodium EDTA, Propylparaben, Sodium Hyaluronate and Sodium Hydroxide.)
10. Zenieva produced by Rivers Edge Pharmaceuticals
11. Prumyx produced by Prugen Pharmaceuticals (similar to Mimyx)
12. Pruvel produced by Prugen Pharmaceuticals

Exemplary Components of the Aqueous Phase

Each of the aforementioned barrier repair compositions (for example, a barrier repair emulsion or a barrier repair formulation) comprises an aqueous phase. The components described below may be present in the aqueous phase of any one of the aforementioned barrier repair compositions.

In certain embodiments, the invention relates to any one of the aforementioned barrier repair compositions, wherein the vehicle comprises water.

In certain embodiments, the invention relates to any one of the aforementioned barrier repair compositions, wherein the vehicle is present in an amount from about 55% to about 85% by weight of the emulsion. In certain embodiments, the vehicle is present in an amount from about 60% to about 80% by weight of the emulsion. In certain embodiments, the vehicle is present in an amount of about 60%, about 62%, about 64%, about 66%, about 68%, about 70%, about 72%, about 74%, about 76%, about 78%, or about 80% by weight of the emulsion.

In certain embodiments, the invention relates to any one of the aforementioned barrier repair compositions, wherein water is present in an amount from about 55% to about 85% by weight of the emulsion. In certain embodiments, water is present in an amount from about 60% to about 80% by weight of the emulsion. In certain embodiments, water is present in an amount of about 60%, about 62%, about 64%, about 66%, about 68%, about 70%, about 72%, about 74%, about 76%, about 78%, or about 80% by weight of the emulsion.

In certain embodiments, the invention relates to any one of the aforementioned barrier repair compositions, wherein the second moisturizer or second emollient is selected from the group consisting of white petrolatum, lactic acid, glycerol, propylene glycol, butylene glycol, sodium PCA, sodium hyaluronate, Carbowax 200, Carbowax 400, Carbowax 800, PPG-15 stearyl ether, lanolin alcohol, lanolin, lanolin derivatives, cholesterol, petrolatum, isostearyl neopentanoate, octyl stearate, mineral oil, isocetyl stearate, myristyl myristate, octyl dodecanol, 2-ethylhexyl palmitate (octyl palmitate), dimethicone, phenyl trimethicone, cyclomethicone, C₁₂-C₁₅ alkyl benzoates, dimethiconol, propylene glycol, Theobroma Graniflorum seed butter, ceramides, hydroxypropyl bispalmitamide MEA, hydroxypropyl bislauramide MEA, hydroxypropyl bisisostearamide MEA, 1,3-bis-(N-2-(hydroxyethyl)stearoylamino)-2-hydroxypropane, bis-hydroxyethyl tocopherylsuccinoylamido hydroxypropane, and dicaprylate/dicaprate, and combinations/mixtures thereof.

In certain embodiments, the invention relates to any one of the aforementioned barrier repair compositions, wherein the second moisturizer or second emollient is selected from the group consisting of glycerol, propylene glycol, sodium hyaluronate, and combinations/mixtures thereof.

In certain embodiments, the invention relates to any one of the aforementioned barrier repair compositions, wherein the second moisturizer or second emollient is present in an amount from about 5% to about 15% by weight of the emulsion. In certain embodiments, the second moisturizer or second emollient is present in an amount from about 7% to about 13% by weight of the emulsion. In certain embodiments, the second moisturizer or second emollient is present in an amount of about 7%, about 8%, about 9%, about 10%, or about 11% by weight of the emulsion.

In certain embodiments, the invention relates to any one of the aforementioned barrier repair compositions, wherein the second moisturizer or second emollient comprises propylene glycol. In certain embodiments, the propylene glycol is present in an amount from about 1.2% to about 3.8% by weight of the emulsion. In certain embodiments, the propylene glycol is present in an amount from about 1.5% to about 3.5% by weight of the emulsion. In certain embodiments, the propylene glycol is present in about 1.5%, about 2%, about 2.5%, about 3%, or about 3.5% by weight of the emulsion.

In certain embodiments, the invention relates to any one of the aforementioned barrier repair compositions, wherein the second moisturizer or second emollient comprises glycerol. In certain embodiments, the glycerol is present in an amount from about 4% to about 11% by weight of the emulsion. In certain embodiments, the glycerol is present in an amount from about 6% to about 9% by weight of the emulsion. In certain embodiments, the glycerol is present in about 6%, about 6.5%, about 7%, about 7.5%, about 8%, about 8.5%, or about 9% by weight of the emulsion.

In certain embodiments, the invention relates to any one of the aforementioned barrier repair compositions, wherein the second moisturizer or second emollient comprises sodium hyaluronate. In certain embodiments, the sodium hyaluronate is present in an amount from about 0.05% to about 0.2% by weight of the emulsion. In certain embodiments, the sodium hyaluronate is present in an amount from about 0.1% to about 0.12% by weight of the emulsion. In certain embodiments, the sodium hyaluronate is present in about 0.1%, about 0.09%, about 0.1%, about 0.11%, or about 0.12% by weight of the emulsion.

In certain embodiments, the invention relates to any one of the aforementioned barrier repair compositions, wherein the second antioxidant or second preservative is selected from the group consisting of imidazolidinyl urea, diazolidinyl urea, phenoxyethanol, sodium methyl paraben, methylparaben, ethylparaben, propylparaben, potassium sorbate, sodium benzoate, sorbic acid, benzoic acid, formaldehyde, citric acid, sodium citrate, chlorine dioxide, benzalkonium chloride, benzethonium chloride, cetrimide, dequalinium chloride, cetylpyridinium chloride, phenylmercuric nitrate, phenylmercuric acetate, thimerosal, chlorobutanol, dichlorobenzyl alcohol, phenylethyl alcohol, benzyl alcohol, ascorbic acid and its esters, sodium bisulfite, butylated hydroxytoluene, butylated hydroxyanisole, tocopherols (such as α-tocopherol), tocopheryl acetate, sodium ascorbate/ascorbic acid, ascorbyl palmitate, propyl gallate, disodium EDTA, and sodium citrate, and combinations/mixtures thereof.

In certain embodiments, the invention relates to any one of the aforementioned barrier repair compositions, wherein the second antioxidant or second preservative is selected from the group consisting of methylparaben, propylparaben, disodium EDTA, and combinations/mixtures thereof.

In certain embodiments, the invention relates to any one of the aforementioned barrier repair compositions, wherein the second antioxidant or second preservative is present in an amount from about 0.2% to about 0.8% by weight of the emulsion. In certain embodiments, the first antioxidant or first preservative is present in an amount from about 0.4% to about 0.6% by weight of the emulsion. In certain embodiments, the first antioxidant or first preservative is present in an amount of about 0.4%, about 0.45%, about 0.5%, about 0.55%, or about 0.6% by weight of the emulsion.

In certain embodiments, the invention relates to any one of the aforementioned barrier repair compositions, wherein the second antioxidant or second preservative comprises methylparaben. In certain embodiments, the methylparaben is present in an amount from about 0.1% to about 0.5% by weight of the emulsion. In certain embodiments, the methylparaben is present in an amount from about 0.2% to about 0.4% by weight of the emulsion. In certain embodiments, the methylparaben is present in about 0.2%, about 0.2%, about 0.3%, about 0.35%, or about 0.4% by weight of the emulsion.

In certain embodiments, the invention relates to any one of the aforementioned barrier repair compositions, wherein the second antioxidant or second preservative comprises propylparaben. In certain embodiments, the propylparaben is present in an amount from about 0.05% to about 0.2% by weight of the emulsion. In certain embodiments, the propylparaben is present in an amount from about 0.1% to about 0.12% by weight of the emulsion. In certain embodiments, the propylparaben is present in about 0.1%, about 0.09%, about 0.1%, about 0.11%, or about 0.12% by weight of the emulsion.

In certain embodiments, the invention relates to any one of the aforementioned barrier repair compositions, wherein the second antioxidant or second preservative comprises disodium EDTA. In certain embodiments, the disodium EDTA is present in an amount from about 0.05% to about 0.2% by weight of the emulsion. In certain embodiments, the disodium EDTA is present in an amount from about 0.1% to about 0.2% by weight of the emulsion. In certain embodiments, the disodium EDTA is present in about 0.1%, about 0.09%, about 0.1%, about 0.11%, or about 0.12% by weight of the emulsion.

In certain embodiments, the invention relates to any one of the aforementioned barrier repair compositions, wherein pH adjuster is selected from the group consisting of citric acid, sodium hydroxide, and sodium phosphate, and combinations/mixtures thereof.

In certain embodiments, the invention relates to any one of the aforementioned barrier repair compositions, wherein the pH adjuster comprises sodium hydroxide.

In certain embodiments, the invention relates to any one of the aforementioned barrier repair compositions, wherein the pH of the emulsion is from about 4.0 to about 7.5. In certain embodiments, the invention relates to any one of the aforementioned compositions, wherein the pH of the emulsion is from about 4.5 to about 6.5. In certain embodiments, the invention relates to any one of the aforementioned compositions, wherein the pH of the emulsion is about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, or about 7.0.

Exemplary Propellants

Each of the aforementioned barrier repair formulations comprises a propellant. The propellants described below may be present in any one of the aforementioned barrier repair formulations.

In certain embodiments, the invention relates to any one of the aforementioned barrier repair formulations, wherein the propellant is selected from the group consisting of 1,1,1,2-tetrafluoroethane, 1,1,1,2,3,3,3-heptafluoropropane, and combinations/mixtures thereof.

In certain embodiments, the invention relates to any one of the aforementioned barrier repair formulations, wherein the propellant is present in an amount from about 3% to about 20% by weight of the formulation. In certain embodiments, the invention relates to any one of the aforementioned formulations, wherein the propellant is present in an amount from about 5% to about 18% by weight of the formulation. In certain embodiments, the invention relates to any one of the aforementioned formulations, wherein the propellant is about 5%, about 6%, about 7%, about 8%, about 9%, about 10%, about 11%, about 12%, about 13%, about 14%, about 15%, about 16%, about 17%, or about 18% by weight of the formulation.

Exemplary Purge Gases

Each of the aforementioned barrier repair formulations comprises a purge gas. The purge gases described below may be present in any one of the aforementioned barrier repair formulations.

In certain embodiments, the invention relates to any one of the aforementioned barrier repair formulations, wherein the purge gas is selected from the group consisting of nitrogen and argon. In certain embodiments, the invention relates to any one of the aforementioned formulations, wherein the purge gas is argon.

In certain embodiments, the invention relates to any one of the aforementioned barrier repair formulations, wherein the purge gas is present in an amount from about 0.4% to about 6% by weight of the formulation. In certain embodiments, the invention relates to any one of the aforementioned formulations, wherein the purge gas is present in an amount from about 0.8% to about 5% by weight of the formulation. In certain embodiments, the invention relates to any one of the aforementioned formulations, wherein the purge gas is about 0.8%, about 1%, about 1.2%, about 1.4%, about 1.6%, about 1.8%, about 2%, about 2.2%, about 2.5%, about 2.6%, about 2.8%, about 3%, about 3.2%, about 3.4%, about 3.6%, about 3.8%, about 4%, about 4.2%, about 4.4%, about 4.6%, about 4.8% or about 5% by weight of the formulation.

Exemplary Properties of Barrier Repair Compositions

In certain embodiments, the invention relates to any one of the aforementioned barrier repair compositions that, upon application to the skin of an affected subject, are non-irritating.

In certain embodiments, the invention relates to any one of the aforementioned barrier repair compositions that, upon application to the skin of an affected subject, are well-tolerated.

In certain embodiments, the invention relates to any one of the aforementioned barrier repair compositions that, upon application to the skin of an affected subject, are non-cytotoxic.

In certain embodiments, the invention relates to any one of the aforementioned barrier repair compositions that, upon application to the skin of an affected subject, are weakly sensitizing. In certain embodiments, the invention relates to any one of the aforementioned compositions that, upon application to the skin of an affected subject, are non-sensitizing.

In certain embodiments, the invention relates to any one of the aforementioned barrier repair compositions that, upon application to the skin of an affected subject, do not produce edema or erythema. In certain embodiments, the invention relates to any one of the aforementioned barrier repair compositions that, upon application to the skin of an affected subject, do not produce edema or erythema after about 24, about 48, or about 72 hours.

In certain embodiments, the invention relates to any one of the aforementioned barrier repair compositions that, upon application to the skin of an affected subject, moisturize the skin. In certain embodiments, the invention relates to any one of the aforementioned barrier repair compositions that, upon application to the skin of an affected subject, improve skin moisturization at least about 6 hours after application.

In certain embodiments, the invention relates to any one of the aforementioned barrier repair compositions that, upon application to the skin of an affected subject, increase hydration of the skin. In certain embodiments, the invention relates to any one of the aforementioned barrier repair compositions that, upon application to the skin of an affected subject, increase skin hydration at least about 6 hours after application.

In certain embodiments, the invention relates to any one of the aforementioned barrier repair compositions that, upon application to the skin of an affected subject, reduce transepidermal water loss. In certain embodiments, the invention relates to any one of the aforementioned barrier repair compositions that, upon application to the skin of an affected subject, reduce transepidermal water loss within about 1 hour of application.

In certain embodiments, the invention relates to any one of the aforementioned barrier repair formulations that, upon expulsion from an aerosol container, forms a foam. In certain embodiments, the foam is temperature-stable. In certain embodiments, the foam is time-stable. In certain embodiments, the density of the foam is from about 0.05 to about 0.5 g/cm³. In certain embodiments, the density of the foam is about 0.14 g/cm³.

### Exemplary Kits

In one embodiment, the invention relates to a kit, wherein the kit comprises any one of the above-mentioned bleach compositions; and any one of the above-mentioned barrier repair compositions.

### Exemplary Methods of Use

In one embodiment, the present invention relates to a method of treating a condition of a subject in need thereof, comprising the steps of:

applying to an affected area of the subject an effective amount of any one of the above-mentioned bleach compositions; and

applying to the affected area of the subject an effective amount of any one of the above-mentioned barrier repair compositions.

In one embodiment, the present invention relates to a method of treating a condition of a subject in need thereof, comprising the steps of:

applying to an affected area of the subject an effective amount of any one of the above-mentioned bleach compositions; and

applying to the affected area of the subject an effective amount of any one of the above-mentioned barrier repair compositions,

thereby treating and moisturizing the affected area.

In one embodiment, the present invention relates to a method of treating a condition of a subject in need thereof, comprising the steps of:

applying to an affected area of the subject an effective amount of any one of the above-mentioned bleach compositions; and

applying to the affected area of the subject an effective amount of any one of the above-mentioned barrier repair compositions,

thereby treating and hydrating the affected area.

In one embodiment, the present invention relates to any one of the above-mentioned methods, wherein the condition is atopic dermatitis, contact dermatitis, xerotic eczema, seborrhoeic dermatitis, psoriasis, dyshidrosis, discoid eczema, venous eczema, dermatitis herpetiformis, neurodermatitis, autoeczematization, herpes simplex I, other topical herpes or viral infections, common cold sores and fever blisters, ringworm, impetigo, or other viral, fungal, or bacterial infections of the skin. In one embodiment, the condition is a heavily contaminated or infected wound. In one embodiment, a heavily contaminated wound is understood by those of ordinary skill in the art to mean a wound that is heavily contaminated by micro-organisms, but not clinically infected. Such wounds are often characterized by a prolonged period of inflammation, as well as a delay in wound healing or repair. In one embodiment, heavily infected wounds are understood by those of ordinary skill in the art to mean wounds with a bioburden greater than 10⁵ micro-organisms per gram of tissue.

In one embodiment, the present invention relates to any one of the above-mentioned methods, wherein the condition is eczema.

In one embodiment, the present invention relates to any one of the above-mentioned methods, wherein the condition is atopic dermatitis. In one embodiment, the present invention relates to any one of the above-mentioned methods, wherein the condition is atopic dermatitis with more than about 10% body surface area (BSA) involvement. In one embodiment, the present invention relates to any one of the above-mentioned methods, wherein the condition is atopic dermatitis with up to about 80% body surface area (BSA) involvement.

In one embodiment, the present invention relates to any one of the above-mentioned methods, wherein the subject is human.

In one embodiment, the present invention relates to the above-mentioned method, wherein the affected area of the subject is the face, earlobes, neck, scalp, genitals, eyelids, palms, fingers, feet, exural (inner) surfaces of joints, extensor aspects of joints, or any combination thereof. In one embodiment, the present invention relates to the above-mentioned method, wherein the affected area of the subject is the face. In one embodiment, the present invention relates to the above-mentioned method, wherein the affected area of the subject is the exural surfaces of elbows or knees. In one embodiment, the present invention relates to the above-mentioned method, wherein the affected area of the subject is the extensor aspects of wrists, elbows, ankles, or knees.

In one embodiment, the present invention relates to any one of the above-mentioned methods, wherein the bleach composition is applied once daily.

In one embodiment, the present invention relates to any one of the above-mentioned methods, wherein the bleach composition is applied twice daily.

In one embodiment, the present invention relates to any one of the above-mentioned methods, wherein the barrier repair composition is applied once daily.

In one embodiment, the present invention relates to any one of the above-mentioned methods, wherein the barrier repair composition is applied twice daily.

In one embodiment, the present invention relates to any one of the above-mentioned methods, wherein the barrier repair composition is applied to the affected area from about 1 minute to about 30 minutes after the bleach composition is applied. In one embodiment, the present invention relates to any one of the above-mentioned methods, wherein the barrier repair composition is applied to the affected area about 1 min, about 2 min, about 3 min, about 4 min, about 5 min, about 6 min, about 7 min, about 8 min, about 9 min, about 10 min, about 15 min, about 20 min, about 25 min, or about 30 min after the bleach composition is applied.

In one embodiment, the invention relates to a method of disinfecting an intact skin site prior to a surgical or invasive procedure.

### Exemplary Constituents of Compositions

Exemplary identities of various constituents of the compositions of the present invention are described below.

### 1. Propellants

In one embodiment, the propellant is a HFA or a mixture of one or more hydrofluorocarbons. Suitable hydrofluorocarbons include 1,1,1,2-tetrafluoroethane (HFA 134a); 1,1,1,2,3,3,3-heptafluoropropane (HFA 227); and mixtures and admixtures of these and other HFAs that are currently approved or may become approved for medical use are suitable. Hydrocarbon as well as chlorofluorocarbon (CFC) propellants can also be used in the present invention.

There are a number of conceivable choices of propellants for a hypochlorite formulation (foam or spray) or a barrier repair formulation (foam or spray), including, but not limited to, CFCs, hydrocarbons, compressed gases, and hydrofluoroalkanes (HFAs). The Montreal Protocol has banned the use of CFCs (chlorofluorocarbons) due to their ability to deplete the ozone layer. Montreal Protocol on Substances that Deplete the Ozone Layer, United Nations Environmental Programme, 1987. Alternatively, hydrocarbon propellants demonstrate very low reactivity and good resistance to free-radical attack. However, hydrocarbon propellants are highly flammable and it would be undesirable and hazardous to combine these propellants with hypochlorite salts, strong oxidizers, in an aerosol system. The chemical classes of “oxidizer” and “flammable” are known to be incompatible. Finally, compressed inert gases, such as nitrogen and carbon dioxide, can be used as an aerosol propellant. While offering good chemical stability due to their non-reactivity, they may be unable to deliver consistent product delivery throughout the life of the aerosol can due to their high vapor pressures. Another option is HFAs. These propellants are pharmaceutically acceptable, generally non-reactive, and ozone-friendly.

### 2. Hypochlorite Salts

A variety of salts of hypochlorous acid (HOCl), both monovalent and divalent, may be present in a composition. These include sodium hypochlorite, potassium hypochlorite, calcium hypochlorite, magnesium hypochlorite, lithium hypochlorite, and copper(I) or copper(II) hypochlorite.

### 3. Other Active Agents

One or more additional active agents may be present in the composition. These include any material that has a desired effect when applied topically to a mammal, particularly a human. Suitable classes of active agents include, but are not limited to, antibiotic agents, antimicrobial agents, anti-acne agents, antibacterial agents, antifungal agents, antiviral agents, steroidal anti-inflammatory agents, non-steroidal anti-inflammatory agents, anesthetic agents, antipruriginous agents, antiprotozoal agents, anti-oxidants, antihistamines, vitamins, and hormones.

3.1 Antibiotics

Representative antibiotics include, without limitation, octopirox, erythromycin, zinc, tetracyclin, triclosan, azelaic acid and its derivatives, phenoxy ethanol and phenoxy proponol, ethyl acetate, clindamycin and meclocycline; sebostats such as flavinoids; alpha and beta hydroxy acids; and bile salts, such as scymnol sulfate and its derivatives, deoxycholate and cholate. The antibiotic can be an antifungal agent. Suitable antifungal agents include, but are not limited to, clotrimazole, econazole, ketoconazole, itraconazole, miconazole, oxiconazole, sulconazole, butenafine, naftifine, terbinafine, undecylinic acid, tolnaftate, and nystatin.

3.2 Non-Steroidal Anti-Inflammatory Agents

Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac, fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed, as well as the dermatologically acceptable salts and esters of these agents.

3.3 Steroidal Anti-Inflammatory Agents

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

In certain embodiments, steroids may be excluded from the compositions of the invention.

3.4 Anesthetics

Suitable anesthetics include the aminoacylanilide compounds such as lidocaine, prilocaine, bupivacaine, levo-bupivacaine, ropivacaine, mepivacaine and related local anesthetic compounds having various substituents on the ring system or amine nitrogen; the aminoalkyl benzoate compounds, such as procaine, chloroprocaine, propoxycaine, hexylcaine, tetracaine, cyclomethycaine, benoxinate, butacaine, proparacaine, butamben, and related local anesthetic compounds; cocaine and related local anesthetic compounds; amino carbonate compounds such as diperodon and related local anesthetic compounds; N-phenylamidine compounds such as phenacaine and related anesthetic compounds; N-aminoalkyl amide compounds such as dibucaine and related local anesthetic compounds; aminoketone compounds such as falicaine, dyclonine and related local anesthetic compounds; and amino ether compounds such as pramoxine, dimethisoquien, and related local anesthetic compounds; and para-amino benzoic acid esters such as benzocaine. Other suitable local anesthetics include ketocaine, dibucaine, amethocaine, propanacaine, and propipocaine.

3.5 Antimicrobial Agents

Suitable antimicrobial agents include, but are not limited to, antibacterial, antifungal, antiprotozoal and antiviral agents, such as beta-lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, metronidazole, pentamidine, gentamicin, kanamycin, lineomycin, methacycline, methenamine, minocycline, neomycin, netilmicin, streptomycin, tobramycin, and miconazole. Also included are tetracycline hydrochloride, famesol, erythromycin estolate, erythromycin stearate (salt), amikacin sulfate, doxycycline hydrochloride, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, triclosan, octopirox, nystatin, tolnaftate, clotrimazole, anidulafungin, micafungin, voriconazole, lanoconazole, ciclopirox and mixtures thereof.

3.6 Keratolytic Agents

Suitable keratolytic agents include, but are not limited to, urea, salicylic acid, papain, sulfur, glycolic acid, pyruvic acid, resorcinol, N-acetylcysteine, retinoids such as retinoic acid and its derivatives (e.g., cis and trans, esters), alpha hydroxy acids, beta hydroxy acids, coal tar, and combinations thereof.

3.7 Other Agents

Suitable other agents include, but are not limited to, skin soothing agents, deodorant agents, antiperspirants, sun screening agents, sunless tanning agents, vitamins, hair conditioning agents, anti-irritants, anti-aging agents, and combinations thereof.

Examples of skin soothing agents include, but are not limited to, allantoin, aloe, avocado oil, green tea extract, hops extract, chamomile extract, colloidal oatmeal, calamine, cucumber extract, and combinations thereof.

Examples of vitamins include, but are not limited to, vitamins A, D, E, K, and combinations thereof.

Examples of sunscreens include, but are not limited to, p-aminobenzoic acid, Avobenzone, Cinoxate, Dioxybenzone, Homosalate, Menthyl anthranilate, Octocrylene, Octyl methoxycinnamate, Octyl salicylate, Oxybenzone, Padimate O, Phenylbenzimidazole sulfonic acid, Sulisobenzone, Titanium dioxide, Trolamine salicylate, Zinc oxide, 4-methylbenzylidene camphor, Methylene Bis-Benzotriazolyl Tetramethylbutylphenol, Bis-Ethylhexyloxyphenol Methoxyphenyl Triazine, Terephthalylidene Dicamphor Sulfonic Acid, Drometrizole Trisiloxane, Disodium Phenyl Dibenzimidazole Tetrasulfonate, Diethylamino Hydroxybenzoyl Hexyl Benzoate, Octyl Triazone, Diethylhexyl Butamido Triazone, Polysilicone-15, and combinations thereof

### 4. Stabilizers

The composition may further include components adapted to improve the stability or effectiveness of the applied composition. These include, but are not limited to, preservatives, buffers, antioxidants, and chelators.

Suitable preservatives for use in the present invention include, but are not limited to: ureas, such as imidazolidinyl urea and diazolidinyl urea; phenoxyethanol; sodium methyl paraben, methylparaben, ethylparaben, and propylparaben; potassium sorbate; sodium benzoate; sorbic acid; benzoic acid; formaldehyde; citric acid; sodium citrate; chlorine dioxide; quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; and alcoholic agents, for example, chlorobutanol, dichlorobenzyl alcohol, phenylethyl alcohol, and benzyl alcohol.

Suitable antioxidants include, but are not limited to, ascorbic acid and its esters, sodium bisulfite, butylated hydroxytoluene, butylated hydroxyanisole, tocopherols (such as α-tocopherol), DL-alpha tocopheryl acetate, sodium ascorbate/ascorbic acid, ascorbyl palmitate, propyl gallate, and chelating agents like ethylenediaminetetraacetic acid (EDTA, e.g., disodium EDTA), citric acid, and sodium citrate.

In addition, combinations or mixtures of these preservatives or anti-oxidants may also be used in the compositions of the present invention.

### 5. Surfactants and Emulsifiers

Many topical compositions contain chemical emulsions which use surface active ingredients (emulsifiers) to disperse dissimilar chemicals in a particular solvent system. For example, most lipid-like (oily or fatty) or lipophilic ingredients do not uniformly disperse in aqueous solvents unless they are first combined with emulsifiers which form microscopic aqueous soluble micelles that contain a lipid-soluble interior and an aqueous-soluble exterior, resulting in an oil-in-water emulsion. In order to be soluble in aqueous media, a molecule must be polar or charged so as to favorably interact with water molecules which are also polar. Similarly, to dissolve an aqueous-soluble polar or charged ingredient in a largely lipid or oil-based solvent, an emulsifier is typically used which forms stable micelles that contain the aqueous-soluble components in the micelle interior while the exterior of the micelle is lipophilic so that it can dissolve in the lipophilic solvent to form a water-in-oil emulsion. It is well known that such emulsions can be destabilized by the addition of salts or other charged ingredients which can interact with the polar or charged portions of the emulsifier within an emulsion micelle. Emulsion destabilization results in the aqueous and lipophilic ingredients separating into two layers, potentially destroying the commercial value of a topical product.

Surfactants suitable for use in the present invention may be ionic or non-ionic. These include, but are not limited to: dicetyl phosphate (1-hexadecanol, hydrogen phosphate), ceteth-10 phosphate (polyethylene glycol hexadecyl ether phosphate), polysorbates (Polysorbate 20, Polysorbate 40, Polysorbate 60, Polysorbate 80), steareth-10, sodium dodecyl sulfate (sodium lauryl sulfate), lauryl dimethyl amine oxide, cetyltrimethylammonium bromide (CTAB), polyethoxylated alcohols, polyoxyethylene sorbitan, octoxynol, N,N-dimethyldodecylamine-N-oxide, hexadecyltrimethylammonium bromide (HTAB), polyoxyl 10 lauryl ether, bile salts (such as sodium deoxycholate or sodium cholate), polyoxyl castor oil, nonylphenol ethoxylate, cyclodextrins, lecithin, dimethicone copolyol, lauramide DEA, cocamide DEA, cocamide MEA, oleyl betaine, cocamidopropyl betaine, cocamidopropyl phosphatidyl PG-dimonium chloride, methylbenzethonium chloride, alkyl polyglucoside, e.g., Triton™ CG-110 (Dow Chemical Co.), ammonium lauroyl sarcosinate, e.g., Perlastan® AL-30 (Struktol, Stow, Ohio), sodium lauroyl sarcosinate, e.g., Perlastan® L-30 (Struktol, Stow, Ohio), and ammonium myristoyl sarcosinate, e.g., Perlastan® M-30 (Struktol, Stow, Ohio). Appropriate combinations or mixtures of such surfactants may also be used according to the present invention.

Many of these surfactants may also serve as emulsifiers in compositions of the present invention.

Other suitable emulsifiers for use in the compositions of the present invention include, but are not limited to, behentrimonium methosulfate-cetearyl alcohol, non-ionic emulsifiers like emulsifying wax, polyoxyethylene oleyl ether, PEG-40 stearate, cetostearyl alcohol (C₁₆-C₁₈ alcohol), ceteareth-12, ceteareth-20, ceteareth-30, ceteareth alcohol, glyceryl stearate, PEG-100 stearate, glyceryl stearate and PEG-100 stearate, steareth-2, steareth-20, and polyoxyethylene monooctadecyl ether, or combinations/mixtures thereof, as well as cationic emulsifiers like stearamidopropyl dimethylamine and behentrimonium methosulfate, or combinations/mixtures thereof.

### 6. Vehicles

Suitable topical vehicles and vehicle components for use with the compositions of the invention are well known in the cosmetic and pharmaceutical arts, and include such vehicles (or vehicle components) as water; organic solvents such as alcohols (particularly lower alcohols readily capable of evaporating from the skin such as ethanol), glycols (such as propylene glycol, butylene glycol, and glycerol), aliphatic alcohols (such as lanolin); mixtures of water and organic solvents (such as water and alcohol), and mixtures of organic solvents such as alcohol and glycerol (optionally also with water); lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile) such as cyclomethicone, demethiconol and dimethicone copolyol; hydrocarbon-based materials such as petrolatum and squalane; and other vehicles and vehicle components that are suitable for administration to the skin, as well as mixtures of topical vehicle components as identified above or otherwise known to the art.

In one embodiment, the compositions of the present invention are oil-in-water emulsions. Liquids suitable for use in formulating compositions of the present invention include water, and water-miscible solvents such as glycols (e.g., ethylene glycol, butylene glycol, isoprene glycol, propylene glycol), glycerol, liquid polyols, dimethyl sulfoxide, and isopropyl alcohol. One or more aqueous vehicles may be present.

In one embodiment, compositions without methanol, ethanol, propanols, or butanols are desirable.

### 7. Moisturizers and Humectants

One of the most important aspects of topical products in general, and cosmetic products in particular, is the consumer's perception of the aesthetic qualities of a product. For example, while petrolatum is an excellent moisturizer and skin product, it is rarely used alone, especially on the face, because it is greasy, sticky, does not rub easily into the skin and may soil clothing. Consumers highly value products which are aesthetically elegant and have an acceptable tactile feel and performance on their skin.

Suitable moisturizers or humectants for use in the compositions of the present invention include, but are not limited to, lactic acid and other hydroxy acids and their salts, glycerol, propylene glycol, butylene glycol, sodium PCA, sodium hyaluronate, hyaluronic acid, Carbowax 200, Carbowax 400, and Carbowax 800.

Suitable humectants for use in the compositions of the present invention include, but are not limited to, 2-ethylhexyl palmitate (hexadecanoic acid, 2-ethylhexyl ester), glycerol, PPG-15 stearyl ether, lanolin alcohol, lanolin, lanolin derivatives, cholesterol, petrolatum, isostearyl neopentanoate, octyl stearate, mineral oil, isocetyl stearate, myristyl myristate, octyl dodecanol, dimethicone, phenyl trimethicone, cyclomethicone, C₁₂-C₁₅ alkyl benzoates, dimethiconol, propylene glycol, and dicaprylate/dicaprate.

In addition, appropriate combinations and mixtures of any of these moisturizing agents or humectants may be used in accordance with the present invention.

### 8. Viscosity Modifiers

Suitable viscosity adjusting agents (i.e., thickening and thinning agents) for use in the compositions of the present invention include, but are not limited to, protective colloids or non-ionic gums such as hydroxyethylcellulose, xanthan gum, and sclerotium gum, as well as magnesium aluminum silicate, silica, microcrystalline wax, beeswax, paraffin, and cetyl palmitate. In addition, appropriate combinations or mixtures of these viscosity adjusters may be utilized according to the present invention.

In one embodiment the viscosity modifier is hydrous sodium lithium magnesium silicate, e.g., Laponite® (Rockwood Additives Limited, Cheshire, UK).

In one embodiment the viscosity modifier is present in a composition of the invention in an amount from about 0.1% to about 6.0% by weight of the concentrate.

### 9. Additional Constituents

Additional constituents suitable for incorporation into the emulsions of the present invention include, but are not limited to: skin protectants, adsorbents, demulcents, moisturizers, buffering agents, sustained release materials, solubilizing agents, skin-penetration agents, abrasives, absorbents, anti-caking agents, anti-static agents, astringents (e.g., witch hazel, alcohol, and herbal extracts such as chamomile extract), binders/excipients, buffering agents, chelating agents, film forming agents, conditioning agents, opacifying agents, and pH adjusters (e.g., citric acid, sodium hydroxide, and sodium phosphate).

Natural fats and oils, other than those listed above, may also be beneficial constituents of the inventive compositions. For example, fats and glyceridic oils from plants, such as Theobroma grandiflorum, may be added.

Suitable fragrances and colors may be used in the compositions of the present invention. Examples of fragrances and colors suitable for use in topical products are known in the art.

Often, one constituent of a composition may accomplish several functions. In one embodiment, the present invention relates to constituents that may act as a lubricant or a skin-penetrating agent. In one embodiment, the multi-functional constituent is socetyl stearate, isopropyl isostearate, isopropyl palmitate, or isopropyl myristate.

### 10. Purging Gases

In one embodiment, the air in the container charged with the composition is replaced by an inert gas. In certain embodiments, the inert gas is selected from the group consisting of argon, nitrogen, and mixtures thereof.

### DEFINITIONS

For convenience, certain terms employed in the specification and appended claims are collected here. These definitions should be read in light of the entire disclosure and understood as by a person of skill in the art.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The phrase “or,” as used herein in the specification and in the claims, should be understood to mean “either or both” of the elements so conjoined, i.e., elements that are conjunctively present in some cases and disjunctively present in other cases. Multiple elements listed with “or” should be construed in the same fashion, i.e., “one or more” of the elements so conjoined. Other elements may optionally be present other than the elements specifically identified by the “or” clause, whether related or unrelated to those elements specifically identified. Thus, as a non-limiting example, a reference to “A or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The invention now being generally described, it will be more readily understood by reference to the following examples, which are included merely for purposes of illustration of certain aspects and embodiments of the present invention, and are not intended to limit the invention.

### EXAMPLES

### Example 1

### Barrier Repair Compositions

In one embodiment, the barrier repair compositions are commercially available creams, lotions, sprays, gels, foams or ointments. Examples of commercially available barrier repair compositions include:

1. Atopiclair produced by Graceway Pharmaceuticals

2. Biafine produced by Ortho Dermatologics

3. Eletone produced by Ferndale Laboratories

4. Epiceram produced by Promius Pharma

5. Hylira produced by Hawthorn Pharmaceuticals

6. Mimyx produced by Stiefel Laboratories

7. Tetrix produced by Valeant Pharmaceuticals

8. Neosalus produced by Quinnova Pharmaceuticals

9. Hylatopic produced by Onset Therapeutics

10. Zenieva produced by Rivers Edge Pharmaceuticals

11. Prumyx produced by Prugen Pharmaceuticals

12. Pruvel produced by Prugen Pharmaceuticals

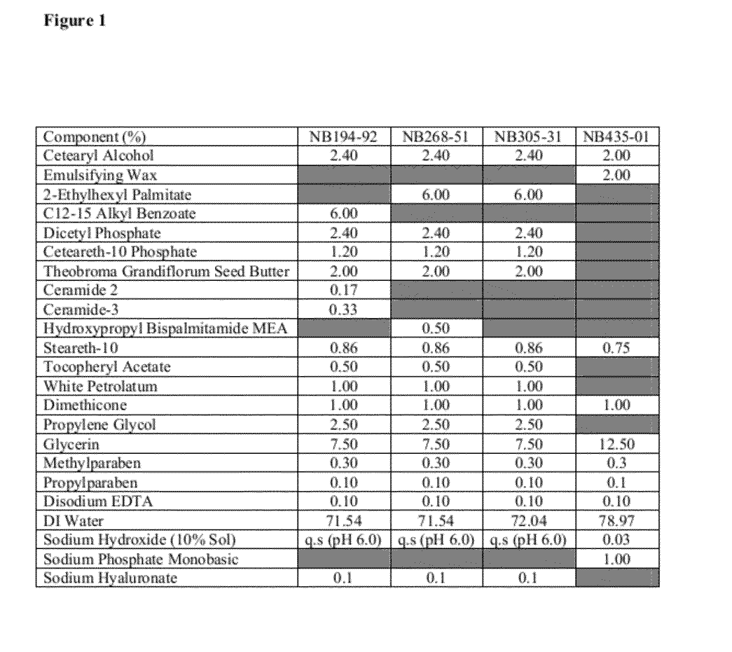

In one embodiment, the barrier repair composition is a drug-free topical cream, lotion, or aerosol foam. Various embodiments of barrier repair emulsions (FIG. 1) were manufactured as outlined below:

### Step 1: Oil Phase Preparation

1. Charged surfactants/co-surfactants and emollients into a Stainless Steel tank and heated to 75-80° C.

### Step 2: Aqueous Phase Preparation

1. Charged purified water (Part A) and humectants into a Stainless Steel tank and heated to 75-80° C.
2. Charged and dissolved preservatives and buffer salts while mixing.
3. Continued mixing until a clear solution was obtained while maintaining a temperature of 75-80° C.

### Step 3: Post-Emulsion Phase Preparation

1. Charged purified water (Part B) into stainless steel tank.

2. Dissolved Sodium Hyaluronate while mixing.

### Step 4: Final Emulsion Formation

1. Added product of Step 1 to product of Step 2 while high shear mixing at 75-80° C.
2. Cooled the emulsion with an outside cold water jacket to 45-50° C. while high shear mixing.
3. Discontinued high shear mixing. Started low shear mixing and continued cooling with cold water jacket to form the vehicle emulsion.
4. When the temperature of the vehicle emulsion was 37° C., added product of Step 3 and continued mixing until uniform.
5. Cooled to 27-32° C. Adjusted to final pH with Sodium Hydroxide and final volume with DI water. Mixed until uniform.

Following manufacturing of the barrier repair emulsion, the finished barrier repair emulsion may be filled into aerosol cans to be dispensed as a foam as outlined below.

1. Aerosol cans are cleaned with compressed air and vacuum.

2. Emulsion is filled into cans.

3. Valves are placed onto the cans.

4. Cans are crimped and hydrofluorcarbon propellant is charged.

5. The aerosol can valve and dip-tube is purged with argon gas.

In certain embodiments, propellant concentrations may range from about 8 to about 15% by weight of packaged product, argon concentrations may range from about 0.8 to about 4.0% by weight of packaged product.

Alternatively the barrier repair emulsion may be packaged in bottles, jars, or tubes to be dispensed as a lotion or cream.

### Example 2

### Change in Transepidermal Water Loss

The TEWL value is a measure of the rate of water lost through the skin (g/h·m²) and is an estimate of the skin's ability to retain moisture. It is an index of the extent of possible damage of the skin's water-barrier function. Because water loss through the skin normally occurs by passive diffusion through the epidermis, higher TEWL values indicate greater water loss and are consistent with increased damage of the barrier function of the stratum corneum such as may occur from atopic dermatitis.

The measurement of the water evaporation is based on the diffusion principle in an open chamber

dw/dt=−D×A×dp/dm

where: A=surface area (m²), w=water transported (g), t=time (h), D=diffusion constant (=0.0877 g/m·h·mm Hg), p=vapor pressure of the atmosphere (mm Hg) and m=distance from skin surface to point of measurement.

### Study Materials and Instrumentation

Tewameter® TM 300 (Courage and Khazaka) Serial Number: 05264788

Scotch® Tape

Kimwipes®

Pre-moistened towelettes—BD Alcohol Swabs (Isopropyl Alcohol, 70% v/v)

Indelible Marker/Marking Template

### Test Procedure

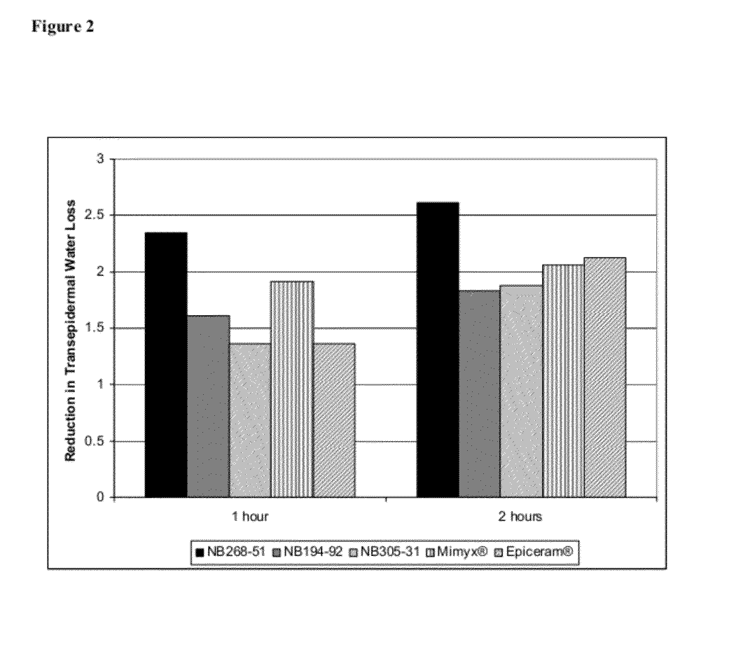

1. Cleaned volar surfaces of both left and right arm with pre-moistened towelette and blotted dry.
2. Marked 2.0×2.0-cm test areas using an indelible marker on volar surface of left and right arms.
3. Measured and recorded the TEWL of all sites two (2) times using the Tewameter. For all measurements, the Tewameter probe was placed on the center of the 2.0×2.0 cm test area. If the center of the test area was a hairy area, the most hairless area within the test site was selected in order to minimize interference with the probe.
4. The transepidermal barrier of the test sites was disrupted by tape stripping or treatment with sodium lauryl sulfate.
5. 15 minutes after barrier disruption, measured the TEWL of all sites two (2) times using the Tewameter. For all measurements, the Tewameter probe was placed on the center of the 2.0×2.0-cm test area. See step 3 for measurement directions.
6. In a randomized, crossover fashion, 135 mg of all test articles were applied to the sites and rubbed-in. Two (2) sites (one on each arm) remain untreated.
7. 1 hour after application, measured the TEWL of all sites (in duplicate).
8. 2 hours after application, measured the TEWL of all sites (in duplicate).
9. Results are depicted in FIG. 2.

### Example 3

### Topical Bleach Compositions

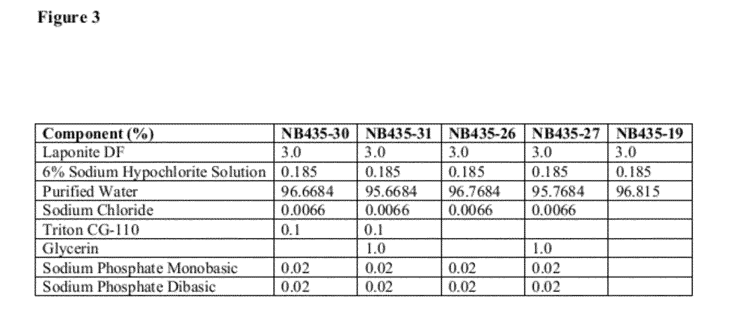

Various bleach compositions were prepared as follows (FIG. 3):
1. Added DI Water, Glycerin, Sodium Phosphate Monobasic, Sodium Phosphate Dibasic, and Sodium Chloride into a SS or Glass Container and mixed for 5 minutes using a prop mixer.
2. Added 6% Sodium Hypochlorite and mixed for 5 minutes using a prop mixer.
3. Began homogenizing with the Silverson. While homogenizing, added the Laponite DF very slowly (powdered in). Took care to add at an addition rate which avoided clumping and accumulation on the sides of the container.
4. After all Laponite DF had been added, homogenized using the Silverson for 10 minutes at speed 6 to efficiently disperse the clay.
5. Removed from Silverson and mixed for 10 minutes using the prop mixer.
6. Added Triton CG-110 slowly with mild mixing. Mixed for an additional 10 minutes.
7. Q.S. with DI water to 750 g. Mixed with prop mixer until uniform.
8. Transferred entire contents to storage container and allowed the composition to thicken to a stiff-gel (2-3 hours)—wrapped in foil when finished.

Following manufacturing of the bleach concentrate, the finished bleach concentrate may be filled into aerosol cans to be dispensed as a foam as outlined below.

1. Aerosol cans are cleaned with compressed air and vacuum.

2. Bleach concentrate is filled into cans.

3. Valves are placed onto the cans.

4. Cans are crimped and hydrofluorocarbon propellant is charged.

5. The aerosol can valve and dip-tube is purged with argon gas.

Propellant concentrations in the bleach formulations may range from about 8 to about 15% by weight of packaged product, argon concentrations may range from about 0.8 to about 4.0% by weight of packaged product.

Alternatively the bleach compositions may be packaged in bottles, jars or tubes to be dispensed as a gel.

### Example 4

### Rapid Reduction in Available Hypochlorite Following Exposure to Barrier Repair Composition

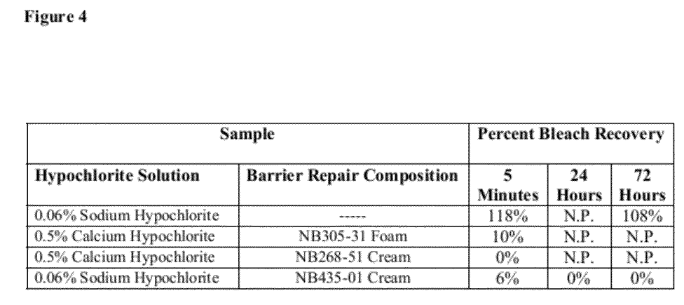

In one embodiment, the barrier repair composition rapidly inactivates hypochlorite it comes into contact with. 3 mL of hypochlorite solution was added to 50 g of deionized water or a barrier repair composition and diluted with 50 mL of deionized water. To this mixture 10 mL acetic acid and 0.5 g potassium iodide were added. The combination was mixed well and then potentiometrically titrated with either 0.1 N or 0.01 N sodium thiosulfate. Each mL of 0.1 N or 0.01 N sodium thiosulfate corresponds to 3.722 or 0.3722 mg sodium hypochlorite (FIG. 4).

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A kit, comprising a bleach composition; and a barrier repair composition.

2. The kit of claim 1, wherein the bleach composition comprises:
an amount of a hypochlorite salt, wherein the amount of the hypochlorite salt is about 0.0001% to about 1.5% by weight of the concentrate;
an amount of a humectant, wherein the amount of the humectant is about 15% to about 35% by weight of the concentrate;
an amount of water, wherein the amount of water is about 60% to about 80% by weight of the concentrate; and
an amount of a stabilizer, wherein the amount of the stabilizer is about 0.5% to about 5.0% by weight of the concentrate.

3. The kit of claim 1, wherein the bleach composition comprises:
an amount of a hypochlorite salt, wherein the amount of the hypochlorite salt is about 0.0001% to about 1.5% by weight of the concentrate;
an amount of a viscosity modifier, wherein the amount of the viscosity modifier is about 0.1% to about 6% by weight of the concentrate;
an amount of a surfactant, wherein the amount of the surfactant is about 0.01% to about 1% by weight of the concentrate;
an amount of water, wherein the amount of water is about 80% to about 99% by weight of the concentrate;
an amount of a stabilizer, wherein the amount of the stabilizer is about 0.01% to about 1.0% by weight of the concentrate;
an amount of a pH modifier, wherein the amount of the pH modifier is from about 0.01% to about 0.06% by weight of the concentrate.

4. The kit of claim 1, wherein the barrier repair composition comprises:
an oil phase, wherein the oil phase comprises an emulsifier or surfactant, a first moisturizer or first emollient, and a first antioxidant or first preservative; and
an aqueous phase, wherein the aqueous phase comprises a vehicle, a second moisturizer or second emollient, a second antioxidant or second preservative, and a pH adjuster.

5. A method of treating a condition of a subject in need thereof, comprising the steps of:
applying to an affected area of the subject an effective amount of a bleach composition; and
applying to the affected area of the subject an effective amount of a barrier repair composition.

6. The method of claim 5, wherein the condition is atopic dermatitis, contact dermatitis, xerotic eczema, seborrhoeic dermatitis, psoriasis, dyshidrosis, discoid eczema, venous eczema, dermatitis herpetiformis, neurodermatitis, autoeczematization, herpes simplex I, other topical herpes or viral infections, common cold sores and fever blisters, ringworm, impetigo, or other viral, fungal, or bacterial infections of the skin.

7. The method of claim 5, wherein the condition is atopic dermatitis.

8. The method of claim 5, wherein the subject is human.

9. The method of claim 8, wherein the affected area of the subject is the face, earlobes, neck, scalp, genitals, eyelids, palms, fingers, feet, exural (inner) surfaces of joints, extensor aspects of joints, or any combination thereof.

10. The method of claim 5, wherein the bleach composition is applied once daily.

11. The method of claim 5, wherein the bleach composition is applied twice daily.

12. The method of claim 5, wherein the barrier repair composition is applied once daily.

13. The method of claim 5, wherein the barrier repair composition is applied twice daily.

14. The method of claim 5, wherein the barrier repair composition is applied to the affected area from about 1 minute to about 30 minutes after the bleach composition is applied.

15. The method of claim 5, wherein the barrier repair composition is applied to the affected area about 1 min, about 2 min, about 3 min, about 4 min, about 5 min, about 6 min, about 7 min, about 8 min, about 9 min, about 10 min, about 15 min, about 20 min, about 25 min, or about 30 min after the bleach composition is applied.
